(19) United States
(12) Patent Application Publication
(10) Pub. No.: US 2009/0142839 A1
(43) Pub. Date: Jun. 4, 2009

(54) Inducing Premature Senescence to Stabilize Stem Cell Feeder Layer Cells

(75) Inventor: Thomas Primiano, Monona, WI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SHILOH LABORATORIES, INC., Monona, WI (US)

(21) Appl. No.: 12/115,451

(22) Filed: May 5, 2008

Related U.S. Application Data
(60) Provisional application No. 60/916,269, filed on May 4, 2007.

(51) Int. Cl. C12N 5/08; C12N 15/00; C12N 5/00

(52) U.S. Cl. 435/455; 435/366; 435/404

(57) ABSTRACT

The present invention provides stem cell feeder layer cell lines that contain are readily triggered to differentiation. The expression vector encodes the senescence-triggering factors (STFs) consisting of Cip/Kip, INK4A, Cy protein or ankyrin-binding protein motifs. Each expression vector also contains an inducible transcription regulation element for conditional expression of the STFs.

### TECHNICAL FIELD

The invention relates to stem cell lines useful in the therapeutic relief of disease by replacing the function of diseased native cells. This invention pertains to stem cells of any origin, embryonic, fetal or adult. For this invention, the feeder layer cells that aid stem cell growth are cultured and engineered to contain a transcriptional regulatory system controlling the expression of senescence-triggering factors (STFs). The STFs are induced chemically to produce premature senescence of the engineered stem cells in situ. The premature senescence phenotype affords stability, increased cellular volume, and resistance to molecular triggers of cell death.

### BACKGROUND OF THE RELATED ART

### 1. Overview of Stem Cell Biology

A stem cell is defined by two properties. First, it is a cell that can divide indefinitely, producing a population of identical offspring. Second, stem cells can, on cue, undergo an asymmetric division to produce two dissimilar daughter cells. One is identical to the parent and continues to contribute to the original stem cell line. The other varies in some way. This cell contains a different set of genetic instructions (resulting in an alternative pattern of gene expression) and is characterized by a reduced proliferative capacity and more restricted developmental potential than its parent. Eventually a stem cell becomes known as a “progenitor” or “precursor” cell, committed to producing one or a few terminally differentiated cells such as neurons or muscle cells.

The different types of stem cell populations can be illustrated by considering the earliest stages of embryogenesis. Soon after fertilization, the haploid nuclei of the egg and sperm merge to form a single nucleus with the diploid number of chromosomes. The zygote divides and its progeny also divide several times thereafter to form a compact ball of cells called the morula (likened in appearance to a mulberry). Each of the 32-128 cells in the morula is totipotent in that each one can give rise to all cell types in the embryo plus all of the extraembryonic tissues necessary for implantation in the uterine wall. These cells are also at the center of preimplantation genetic testing.

As the morula is swept along the oviduct, the cells continue to proliferate and the morula enlarges to form a hollow sphere called a blastocyst (or blastula). During the final days in the oviduct and the first days in the uterus, a few cells delaminate from the surface layer of the blastula to form an inner cell mass (ICM) within the cavity. This cluster of cells is the source of embryonic stem cells. It is important to emphasize that the ICM forms prior to implantation. Blastocysts created in vitro contain an ICM even though the embryo was created and maintained in a test tube. It is possible to isolate cells from the ICM of human blastocysts and grow them in tissue culture, using techniques first developed 20 years ago for the manipulation of mouse embryos. Cells dissociated from the ICM are pluripotent in that they can become any of the hundreds of cell types in the adult body. They are not totipotent because they do not contribute to extraembryonic membranes or the formation of the placenta. The time from fertilization to implantation in the uterine wall is approximately 14 days in humans. Soon after implantation, the blastocyst invaginates, much like a finger pressing into a round rubber balloon. A critical series of cell movements known as gastrulation results in the formation of the three germ layers of the developing embryo: the ectoderm, the endoderm, and the mesoderm. The basic plan of the human body is laid out during this remarkable process as the fate of many cells is determined: the endoderm gives rise to the vasculature and blood-forming organs; the mesoderm produces muscle; and the ectoderm gives rise to the skin and the nervous system.

Stem cells are present in each of the three germ layers. The spectrum of offspring from these stem cells is more restricted than that of cells derived from the ICM, so they are described as multipotent rather than pluripotent. Cells in one germ layer breed true; they do not ordinarily transdifferentiate to form derivatives of other germ layers. Indeed, there is strong evidence for a restriction of developmental potential with time throughout embryogenesis [1, 2]. However, the plasticity of adult stem cells is an issue of great interest, and it merits further investigation.

### Stem Cells in Adult Tissues

Stem cells have been identified in adult tissues including skin, intestine, liver, brain, and bone marrow. Bone marrow stem cells have been studied most extensively because a variety of cell surface and genetic markers have helped delineate various stages of their differentiation during hematopoiesis.

But there are several drawbacks that, a priori, make adult stem cells less attractive than embryonic stem cells as sources for most of the uses described above. It has been difficult to isolate stem cells from adult tissues. The cells are few in number, and it is difficult to keep them proliferating in culture. To date, it appears that cultured adult stem cells give rise to only a limited number of cell types. Finally, they are adult cells and have been exposed to a lifetime of environmental toxins and have also accumulated a lifetime of genetic mutations.

Despite these apparent drawbacks, research on adult stem cells should be pursued vigorously because these problems may be overcome with new techniques and insights. The therapeutic value of partially purified hematopoietic stem cells in repopulating the bone marrow following high-dose chemotherapy is based on the discovery of growth factors that promote the multiplication of blood precursor cells. The same type of information about the differentiation of other types of adult stem cells is needed in order to fully exploit their potential for regenerative medicine.

### Embryonic Stem Cells

The ability of hESCs to proliferate indefinitely in tissue culture and the wide range of cell types to which they give rise make these cells unique. They become even more valuable as new molecules that trigger their differentiation in vivo are discovered. It has proven easier to mimic the normal sequence of development than to reverse this process in an attempt to have cells dedifferentiate.

In 1998, capitalizing on nearly twenty years of experience with mouse embryonic stem cells, scientists at the University of Wisconsin isolated stem cells from the ICM of human blastocysts and grew them in tissue culture for prolonged periods of time ([3]. Under the right conditions, several types of mature cells appeared in the cultures, including nerve cells, muscle cells, bone cells, and pancreatic islet cells. This discovery has led to an explosion of research on hESCs.

### 2. Usefulness of Stem Cells

Results obtained from studies with mouse ESCs (mESCs) raise the possibility that clinical trials with hESCs are not far off. mESCs have been manipulated to become spinal cord motor neurons [4], dopaminergic neurons [5, 6], and many other types of cells. One example must suffice here to emphasize their therapeutic promise. In one of the most thorough and elegant studies published to date, mESCs were induced to differentiate into spinal cord motor neurons by successive exposure to retinoic acid and sonic hedgehog, a protein known to trigger the differentiation of motor neurons in developing embryos [4]. When treated cells were injected into the spinal cord of a chick embryo, they migrated to their proper location in the ventral horn. Some cells sent axons out of the spinal cord to invade the developing limb and form synapses on target muscle fibers. This type of research lends hope to individuals suffering from amyotrophic lateral sclerosis, spinal muscular atrophy, spinal cord injury, and related disorders.

Pancreatic islets have been implanted into patients with type 1 diabetes to restore them to insulin independence [7]. Islet transplantation is successful, when performed according to the Edmonton protocol ({Kim, 2002 #8; Shapiro, 2000 #9}. Likewise, implantation of fetal mesencephalic brain tissue into the brains of patients with Parkinson disease has resulted in measurable improvement in some indices of motor performance (Freed, 2001 #10). Both implantation studies, however, were limited by tissue availability and, in the Parkinson disease study, there were serious side effects (e.g., dyskinesias). Both studies call for further work with hESCs, with the hope of moving to Phase 1 clinical trials.

There is much to learn regarding the use of stem cells for the treatment of disease. One present limitation on using hESCs for treating disease is that they must be grown on feeder layers, heterologous non-stem cells that produce factor(s) necessary for normal hESC growth. These cells are conventionally irradiated mouse embryonic fibroblasts, which function quite reliably in vitro. However, xenobiotic concerns such as mouse retrovirus contamination are a serious drawback to using hESC grown on such murine feeder layers. There is thus a need in the art to identify human cells that can be used as feeder layers in the same way, but without the xenotoxicity concerns. No such human cell has been identified to date.

Additional information about how to keep ESCs dividing until they are called on to differentiate is needed, and more must be learned about the growth factors that influence their differentiation into diverse cell types. Most importantly, stem cell therapy protocols must also be safe as well as effective. This will be greatly facilitated by our understanding of how to turn these cells off in vivo in the event that toxicity develops. In addition, the risk of immune rejection remains a problem. Given the limited genetic diversity of available cell lines, transplantation of stem cell products is subject to the same immune barriers as organ transplantation. At the present time, our only defense against rejection is the administration of long-term immunosuppression therapy, which increases the patients' risk of infection and is associated with nephrotoxicity.

### SUMMARY OF THE INVENTION

In one aspect, the present invention provides human feeder layer cells capable of supporting growth of human stem cells, especially embryonic stem cells, that maintain a euploid chromosomal complement, do not spontaneously differentiate in vitro, have unlimited proliferative capacity, and capable of differentiating into each of the three embryonic cell layers (ectoderm, mesoderm and endoderm). As provided herein, said stem cell feeder layer cells comprise an expression vector that encodes senescence-triggering factors (STFs). Each expression vector also contains an inducible transcription regulation element allowing conditional control of the expression of the STFs.

In one embodiment, the STFs comprise Cip/Kip protein or a biologically-functional fragment thereof. In another embodiment, the STFs comprise an IN4A protein or a biologically-functional fragment thereof. In yet another embodiment, the STFs comprise one or a plurality of Cy motifs. In another embodiment, the STFs comprise one or a plurality of ankyrin-binding motifs. In another embodiment, a combination of STFs is comprised one or a plurality of both ankyrin-binding and Cy motifs.

In yet another embodiment, an expression vector contains a selectable marker, and a multiple cloning site flanked by a polyadenylation signal. The inducible transcription regulation element and polyadenylation site are functionally positioned in an orientation to the senescence-triggering factor to be expressed. The inducible transcription regulation element contains an SV40 or similar viral promoter and functional operator sequences such as lac repressor operator.

The invention also provides methods for producing human stem cell feeder layer cells comprising expression vectors encoding STFs. The invention also provides methods for growing stem cells, particularly human stem cells and specifically including human embryonic, fetal and adult stem cells in the presence of human stem cell feeder layer cells of the invention. The invention also provides methods for growing stem cells, particularly human stem cells and specifically including human embryonic, fetal and adult stem cells in the presence of cell culture media conditioned by growth of human stem cell feeder layer cells of the invention.

Specific preferred embodiments of the present invention will become evident from the following more detailed description of certain preferred embodiments and the claims.

### BRIEF DESCRIPTION OF THE DRAWINGS

FIG. 1 is a diagram showing the p53 and Rb pathways that mediate senescence.

FIG. 2 is a graph showing the numbers of viable cells counted each day during culture. Squares, cells with empty RP Shift vector exposed to doxycycline (2 μM) in culture. Triangles, cells containing STFs induced with doxycycline (2 μM).

FIG. 3 is a photomicrograph showing (Top) cells harboring empty RP Shift vector after 6 days exposure to doxycycline (2 μM) in culture; and (Bottom), cells containing STFs induced for 6 days with doxycycline (2 μM).

FIG. 4 is a graph showing the effects of doxycliclin in inducible cell

FIG. 5 is a photomicrograph showing the morphology of hESC colonies cultured in a variety of methods. Human hESCs were cultured as follows: (A) H7p35 were grown on human fibroblasts cells on gelatin in SR based medium (B) BG01V-Oct4-promoter-E-GFP on human fibroblasts cells on gelatin in BG media (C) BG01V on NHFF240-mRFP/hTERT on gelatin in BG media (D) BG01V on Matrigel in 1:1 BG and fibroblast conditioned BG media, (E/F) BG01V and on RP-Shift feeders in BG media with 1 μM IPTG on gelatin or Kappa-Carrageenan, respectively.

FIG. 6 is a photomicrograph showing Immortalized red fluorescent human foreskin feeder layers support growth of hESCs: BG01VOct4p-EGFP hESCs (B) were plated on gelatin-coated dishes seeded with 100,000 RP Shifted human fibroblast cells. Colonies formed classical elliptical shapes seen with human foreskin feeder layers and maintained Oct4 promoter activity.

FIG. 7 is a photomicrograph showing Growth of ES cell colonies on RP Shift human fibroblast feeder layer cells: BG01VOct4p-EGFP hESCs were plated on gelatin-coated dishes seeded with 100,000 RP Shifted human fibroblast cells. Colonies formed in rounded or elliptical shapes and maintained Oct4 promoter activity and with high density and size on these feeder layer cells.

FIG. 8 is a graph showing. Enhanced Colony formation using RP Shifted Feeder Layer Cells. H7 or BG01VOct4p-EGFP hESCs were plated on gelatin-coated dishes seeded with 100,000 RP Shifted human fibroblast cells or irradiated human fibroblast cells. The number of ES colonies were counted microscopically using an etched P50 cell culture dish.

FIG. 9 is a graph showing Enhanced Colony Size using RP Shifted Feeder Layer Cells. H7 or BG01VOct4p-EGFP hESCs were plated separately on gelatin-coated dishes seeded with 100,000 RP Shifted human fibroblast cells or irradiated human fibroblast cells. The size of ES colonies was determined microscopically using an etched P50 cell culture dish with a micrometer scale. Diameters of 20 separate colonies were measured and standard deviation calculated and plotted.

FIG. 10 is a graph showing Enhanced ES Cell Number using RP Shifted Feeder Layer Cells. H7 or BG01VOct4p-EGFP hESCs were plated separately on gelatin-coated dishes seeded with 100,000 RP Shifted human fibroblast cells or irradiated human fibroblast cells. ES cells were separated from feeder layer cells by digestion with dispase. The number of ES cells was determined microscopically using a hemacytometer. Average numbers of 3 separate cultures are shown with standard deviations.

FIG. 11 is a photomicrograph showing hESC growth on RP Shift-transformed human HT1080 fibrosarcoma cells.

FIG. 12 are photomicrographs of unstained (Panels A, D, G and J) and immunohistochemically-stained BG01V hESCs as disclosed in Example 4.

### DETAILED DESCRIPTION OF CERTAIN EMBODIMENTS

The present invention relates to methods for preparing human feeder layer cells capable of supporting growth of human stem cells, especially embryonic stem cells, that maintain a euploid chromosomal complement, do not spontaneously differentiate in vitro, have unlimited proliferative capacity, and capable of differentiating into each of the three embryonic cell layers (ectoderm, mesoderm and endoderm), wherein said feeder cells are competent for being induced to enter senescence prematurely. In one embodiment, such human feeder layer cells can be used to supporting growth of human stem cells, especially embryonic stem cells, that maintain a euploid chromosomal complement, do not spontaneously differentiate in vitro, have unlimited proliferative capacity, and capable of differentiating into each of the three embryonic cell layers (ectoderm, mesoderm and endoderm) by being switched from a replicative to a productive state commonly known as terminal differentiation

In order to produce a premature senescent state in a cell, embodiments of this invention directly manipulate the progress of the cell cycle. The cell cycle can be divided into four phases:

S (synthetic) phase: the entire genome is duplicated. In this phase cellular energy is used to synthesize enzymes essential for nucleotide synthesis, followed by DNA replication, proof-reading the finished product and correcting detected mistakes.

M (mitotic) phase: parental cell needs energy to compact the entire duplicated genome into chromosomes, to produce a complex protein scaffold, the mitotic spindle, to attach, align and pull apart the duplicated chromosomes. This is followed by cytokinesis, when the cell divides, forming two identical cells.

G1 (gap) phase: the period between M and S phases. The longest phase of the cell cycle. Its length depends on the type of cell as well as its environment. If sufficient nutrition or growth stimulus is not available cells will stop in G1 and not enter S phase. Following exit from M, cells can escape from the cell cycle into a non dividing state; this is usually followed by terminal differentiation.

G2 phase: the period after S phase and before mitosis. Cells generally do not spend much time in this phase, but divide soon after S phase is complete.

Precise coordination of the cell cycle events is required for successful reproduction. Completion of the S phase is essential before the cell passes its genetic material to daughter cells. Any mistake at this stage can be fatal to the progeny, and cells have developed elaborate mechanisms for controlled entry into S and M phases. The paradigm of cell cycle regulation requires orderly execution of cell cycle events, so that the completion of one event is necessary for the beginning of the next. Regulatory feedback controls, that keep the cell cycle from progressing if an essential event does not occur, are called checkpoints.

Genes, responsible for controlled progression of the cell cycle, were first described as cell division cycle (cdc) genes in budding yeast. The majority of cdc genes turned out to be either protein kinases or protein phosphatases; one of the most important among them is protein kinase cdc2. Homologues to cdc2, called cyclin-dependent kinases (CDKs) regulate the cell cycle in higher eukaryotes including humans. CDK-mediated transfer of phosphate groups to target proteins facilitates their activation or repression, which ultimately results in the progress of the cell cycle. Activation of CDKs depends on their association with protein co-factors-cyclins.

Each phase of the cell cycle is characterized by a unique pattern of CDK activity. Eight CDKs have been identified in mammalian cells, and most are active in certain phases of the cell cycle. Thus, progression through G1 depends on the activities of CDK2, CDK3, CDK4, and CDK6, while CDK2 and cdc2 are active in S phase, and cdc2 governs entry and exit into mitosis.

Likewise, cyclins are a group of related proteins that contain a conserved region of homology (the cyclin box) and are expressed in specific phases of the cell cycle. Cyclin levels are rate limiting for the actions of CDKs.

D-type cyclins and cyclin E are expressed in G1 phase of the cycle, when they are involved in regulation of entry into S phase. These cyclins associate with CDK4/6 and CDK2, respectively. There are three types of D cyclins. Cyclin D is expressed early in G1 when quiescent (non-dividing) cells are stimulated by environmental or growth factors to enter the cell cycle. Cyclin D levels remain high as long as mitogen levels are elevated. These proteins are labile (having a half-life of 20 minutes), and their production depends on the continued presence of mitogens. Hence, growth factor or serum deprivation lowers cyclin D levels and halts the cell cycle in G1. Forcing the expression of cyclin D accelerates progression through G1 and its inhibition blocks cells in G1. Cyclin E activity is also required in G1, although following cyclin D activity. Cyclin E activity peaks at the G1-S boundary, and decays as S phase progresses. Cyclin E is regulated transcriptionally by E2F and also by proteolysis, and is required for entry into S phase.

Later cell cycle transitions are mediated by cyclin A and B. Cyclin A associates with CDK2 and cdc2 and its activity is required in S phase and for the G2-M transition. Cyclin B associates with cdc2 and regulates mitotic entry and exit.

Cyclin-dependent kinases (CDKs) are also regulated by phosphorylation and dephosphorylation. The site of CDK activation is a conserved threonine residue within a T loop. The binding of cyclin and the phosphorylation of the CDK move the T loop away from the catalytic site of the enzyme allowing substrate to bind. Conversely, phosphorylation of tyrosine in the N-terminal region inhibits CDK activity. Enzymes that inactivate CDKs by adding phosphate to these tyrosine groups are conserved in many species and are known as wee1 and mik1 kinases. Dephosphorylation of CDKs is mediated by cdc25 phosphatase. A balance of these activities sets a threshold for CDK activation and determines mitotic entry. Thus, CDKs, through their ability to regulate cyclins, play a central role in controlling cell proliferation.

A critical regulator of cell cycle progression in mammalian cells is the family of Rb proteins. Rb proteins undergo phosphorylation during G1, which modifies its interaction with a critical transcription factor E2F. E2F transcription factors are heterodimeric DNA binding proteins composed of one E2F factor and one DP factor that are required for the transcriptional regulation of many proteins needed for S phase progression. There are five known E2F factors, and 3 DP factors. When complexed with Rb, E2F is inactive or may even function as a repressor, thereby silencing E2F-dependent promoters, which, in turn, arrests the cell cycle for a lack of the needed gene products. Phosphorylation of Rb regulates E2F activity. Unphosphorylated Rb avidly binds E2F in early G1, but its phosphorylation at multiple sites lowers its affinity for E2F and releases it to complex with DP in late G1.

The CDKs are responsible for the phosphorylation of Rb. The Rb protein has eight consensus phosphorylation sites and CDKs complexed with cyclin D, E, and A have Rb kinase activities. Cyclin D-CDK 4 has very high affinity for dephosphorylated Rb. Cyclin B-CDK4/6 and cyclin E-CDK2 cooperate to inactivate the E2F binding of Rb. The cyclin D function is not essential in Rb-deficient cell lines, suggesting the function of D-type cyclins of promoting G1 phase progression. Cyclin E-CDK2 may also be necessary for G1/S phase transition.

### CDK Inhibitors (CKIs)

An important aspect of the present invention is the inhibition of cell proliferation. Because CDKs play an essential role in cell proliferation, inhibitors of CDK activity are particularly useful in the compositions and methods of the present invention. All organisms express proteins that directly bind to and inhibit CDK activity. These inhibitors provide another means of cell cycle control in response to diverse stimuli.

Mammalian cells express two classes of CKIs that are distinguished by their CDK targets. The members of the Cip/Kip family of CKIs are universal inhibitors, and INK4 proteins are specific for CDK4/6 inhibition. The Cip/Kip family members are p21, p27, and p57. Over-expression of these gene products blocks cells in G1 phase in culture. They are able to inhibit all cyclin-CDK complexes in vitro. These proteins bind avidly to cyclin-CDK complexes, more so than to the factors separately.

The INK4 family of CKIs includes four structural proteins (p15, p16, p18, and p19), each of which contains four ankyrin repeats. The first member of this family to be identified, p16, was found to be associated with CDK4 in transformed cells and subsequently was identified as a tumor suppressor in familial melanoma. INK4 proteins bind to monomeric CDK4/6 subunits, preventing their association with D-type cyclins, and INK4 proteins also can inhibit the activity of cyclin D-CDK4/6 complexes.

This invention provides methods for inducing premature senescence in cells, particularly human feeder layer cells capable of supporting growth of human stem cells, especially embryonic stem cells, that maintain a euploid chromosomal complement, do not spontaneously differentiate in vitro, have unlimited proliferative capacity, and capable of differentiating into each of the three embryonic cell layers (ectoderm, mesoderm and endoderm). Senescence is defined as the permanent halt in cellular division[8]. Replicative or cellular senescence was observed and proposed as a model for aging at the cellular level over forty years ago[9, 10]. When cells are serially cultured, they undergo a number of rounds of divisions, but as they age, the cells are no longer able to divide. Senescent cells are actually resistant to programmed cell death and some senescent cells have been maintained in their nondividing state for as long as three years [11]. These cells are very much alive and metabolically active, but they do not divide. This nondividing state is irreversible by any biological, chemical, or viral agent. At this stage of terminal nondivision, it has been shown by gene expression that the cells have undergone global changes compared to those of their younger counterparts. The relationship between the changes in gene expression and cellular senescence has not been definitively established, and it is not known whether any or all of the changes cause senescence or whether senescence results in the changes in gene expression. Gene expression changes that could potentially induce senescence include a repression of cell-growth-inducing transcription factors [12]. However, along with this repression of growth inducers is an activation of the cell cycle inhibitors, p21 and p16, which are likely the genes that act to induce cell senescence and in fact are the end products of genetic programs that lead cells to senescence [11].

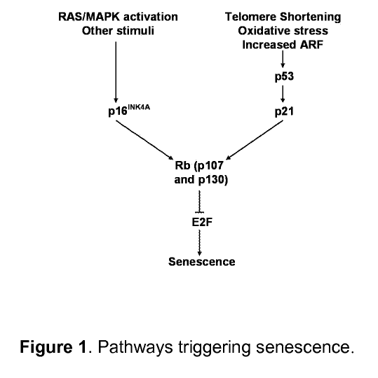

Two separate pathways mediate premature senescence (illustrated in FIG. 1). p53, a regulatory protein that controls cell division [13] and Rb, the central enzyme of cell division, are activated by various stimuli, including telomere shortening, certain forms of DNA damage, and p14ARF expression (which in turn results from oncogene activation). Increased p53 causes a p21-dependent form of growth arrest. Expression of p21 inhibits phosphorylation of Rb family members, resulting in repression of E2F activity that promotes senescence. Expression of p16INK4a is increased by telomere-independent signals, such as MAP kinases. Other stimuli that induce (or inhibit repression of) p16INK4a are not fully characterized. Expression of p16INK4a likewise inhibits Rb phosphorylation with attendant induction of senescence.

Attempts to induce senescence by activating only one of these pathways have yielded results insufficient for long term cell culture. Expression of p16 alone has been shown to produce a reversible phenotype similar to senescence (quiescence) following cell cycle arrest {{Dimri, 2000 #17; Uhrbom, 1997 #18; Vlach, 1996 #19}. These cells overcome cell cycle arrest and begin proliferating in 7-10 days. There are three potential reasons for this reversal of cell cycle inhibition; 1) the cells have stopped producing p16, 2) p16 is inactivated, 3) p16 is degraded. It is unlikely that cells halted synthesis of p16 that is being driven by a powerful eukaryotic promoter. The INK4 family of CKIs includes four structural proteins (p15, p16, p18, and p19, each of which contains four ankyrin repeats [14]. INK4 proteins bind to monomeric CDK4/6 subunits through these ankyrin repeat motifs {Sheaff, 1995 #21; {Serrano, 1993 #22; Kamb, 1994 #23} as shown in TABLE 1, preventing their association with D-type cyclins, and INK4 proteins also can inhibit the activity of cyclin D-CDK4/6 complexes. [14, 15]. Cells that become resistant to p16 may elevate their levels of CDK4 or CDK 6, effectively titrating the p16 produced. Alternatively, INK4 proteins have half-lives of about 20 min in cell culture {{Baldin, 1993 #25; Quelle, 1993 #26}. As disclosed herein, RP Shift expression vectors include one or a multiplicity of Ankyrin motifs, wherein in certain embodiments the multiplicity of Ankyrin motifs are expressed as a peptide multimer of said motifs.

TABLE 1
Ankyrin repeat motifs

ANKYRIN III REGION

| | |
|---|---|
| P16 | RPVHDAAREGFLDTLVVLHRA |
| P19 | SPVHDAARTGFLDTLKVLVEH |
| P18 | AVIHDAARAGFLDTLQTLLEF |
| P15 | RPVHDAAREGFLDTLVVLHRA |

Expression of p21 alone produces a more stable senescence-like phenotype than expression of p16 alone [16, 17], however, overexpression of p21 leads to mitotic catastrophe, a slow form of cell death similar to apoptosis [18]. p21 has two functional domains, an N-terminal CDK binding region, and a carboxy-terminal region that associates with PCNA, a processing factor for DNA polymerase delta [19-21]. It has been proposed that the N-terminal region including the cyclin binding Cy motif of the CIP/KIP family of CDK inhibitors [22] can interact with the cyclins independently of CDK2. The cyclin-binding motifs of p21 are required for the optimal inhibition of cyclin-CDK kinases in vitro and for growth suppression in vivo.

Blocking the turnover of these proteins using an inhibitor of the ubiquitin-targeting proteosome, MG132 [23] sustains the levels of INK4 protein thereby enhancing the cell cycle arrest produced by premature senescence.

Alternatively, it is suggested that fragments of Cip/Kip and INK4 proteins deficient in proteasome targeting sequences are stable in cells by being refractory to proteasome degradation.

An alternative approach to expressing full-length cDNA of p16 and p21 is to express fragments of these genes that may reduce the likelihood of degradation by ubiquitin-targeting or inactivation by binding of cyclin-CDKs. For instance, the cyclin binding motif Cy motif of the CIP/KIP family of CDK inhibitors [22] can interact with the cyclins independently of CDK2. The cyclin-binding motifs of p21 are required for the optimal inhibition of cyclin-CDK kinases in vitro and for growth suppression in vivo. Peptides containing only the N-terminal or C-terminal motif of p21 partially inhibit cyclin-CDK kinase activity in vitro and DNA replication in Xenopus egg extracts. A Cy motif is found near the N terminus of Cdc25A that is separate from the catalytic domain [24]. Mutations in this motif disrupt the association of Cdc25A with cyclin E- or cyclin A-CDK2 in vitro and in vivo and selectively interfere with the dephosphorylation of cyclin E-CDK2. A peptide based on the Cy motif of sequence of p21 competitively disrupts the association of Cdc25A with cyclin-CDKs and inhibits dephosphorylation of the kinase. p21 inhibits Cdc25A-cyclin-CDK2 association and dephosphorylation of CDK2. Conversely, Cdc25A associates with cyclin-CDK and protects it from inhibition by p21. Cdc25A also protects DNA replication in Xenopus egg extracts from inhibition by p21. Thus, cdc25A and p21 compete for binding with cyclin-CDK complexes. The Cy motif sequence is found in many proteins involved in cell cycle dynamics, and the association of cdc25A, p21, cyclins and CDKs is mediated, in part, by the Cy motif. An alignment of Cy motifs from various cell-cycle associated proteins is presented in TABLE 2. As disclosed herein, RP Shift expression vectors include one or a multiplicity of Cy motifs, wherein in certain embodiments the multiplicity of Cy motifs are expressed as a peptide multimer of said motifs.

TABLE 2
Cy motifs

Cy motif

| | | | | | | |
|---|---|---|---|---|---|---|
| E2F1 | K | R | R | L | D | L |
| E2F2 | K | R | K | L | D | L |
| E2F3 | K | R | R | L | E | L |
| p107 | K | R | R | L | F | G |
| p130 | K | R | R | L | F | V |
| Cdo8 | G | R | R | L | V | F |
| Myl1 | P | R | N | i | L | S |
| Cdc25a | P | R | R | L | L | F |
| p57 | C | R | S | L | F | G |
| p27 | C | R | N | L | F | G |
| p21(N) | C | R | R | L | F | G |
| p21(C) | K | R | R | L | I | F |
| HPV16 E1 | K | R | R | L | F | T |
| SSeCKS (1) | L | K | K | L | F | S |
| SSeCKS (2) | L | K | K | L | S | G |
| b3-endonexin | K | R | S | L | K | L |

Other aspects of the invention comprise RP Shift expression vectors comprising one or a multiplicity of Cy motifs and one or a multiplicity of Ankyrin motifs as STFs that can induce senescence in a stem cell feeder layer cell.

In preliminary studies, consensus DNA sequences of senescence-triggering ankyrin repeat similar to the region of p16 (amino acids 1-60), and similar to the Cy motif of p21 (amino acids 1-81), were separately cloned into a nucleotide sequence encoding an alpha helix produced by amino acids 1-40 of the Escherichia coli L7/L12 ribosomal protein (Accession number P02392) {{Bocharov, 1996 #37; SEQ ID No. 1. This peptide-alpha helix encoding nucleic acid was, in turn, cloned into a retroviral expression vector which was transfected with the vesicular stomatitis viral DNA into viral packaging cells using standard Ca-phosphate transfection techniques {Baldin, 1993 #25}. The resulting recombinant retrovirus were used to infect HT1080 E-14 cells that actively produce the convenient marker protein plasminogen activator inhibitor-1 {PAI-1; {Kang, 1998 #38; Wileman, 2000 #39}, and the cells harboring the STFs were selected by antibiotic treatment for which the recombinant retrovirus carried a cognate resistance marker gene.

In growing cultures, senescence must occur after the cells reach an optimal density for supporting growth of human stem cells, especially embryonic stem cells, that maintain a euploid chromosomal complement, do not spontaneously differentiate in vitro, have unlimited proliferative capacity, and capable of differentiating into each of the three embryonic cell layers (ectoderm, mesoderm and endoderm). Proper timing of premature senescence is essential to maximize stem cell support after developing the premature senescent state. This proper timing can be achieved by placing the expression of the STFs under the regulation of an inducible promoter. To satisfy this requirement, a retroviral vector with a modified TetR system was developed [25]. This repressor system must silence expression of the STFs as the culture grows to optimum cell density.

Expression of the STFs was induced by adding doxycycline (a stable derivative of tetracycline) into the media. Following induction, cell proliferation, as monitored by counting cells, was found to be stopped within 24 hours, and proliferation of the cells remained blocked for as long as 30 days. Senescence differs from other forms of growth arrest, such as quiescence, in two important ways. First, senescence in somatic cells is thought to be irreversible, and it therefore represents a specialized form of terminal differentiation. Second, it encompasses certain phenotypic alterations such as characteristic morphological changes and the expression of senescence-associated-β-galactosidase (SA-β-Gal) activity. Recently, senescence has been shown to correlate with the establishment of an unusual form of heterochromatin that is present in discrete nuclear foci [called SA-heterochromatic foci (SAHF)]. In aggregate, these data suggest that senescence results from the durable repression of promoters associated with growth. This repression is enforced by the construction of stable heterochromatin-like complexes, the formation of which is directed in part by hypophosphorylated Rb.

The onset of premature senescence was observed by staining cells for SA-β-gal activity [26]. Cells maintained this phenotype for thirty days. Cells to which empty retroviral vector was infected did not express SA-β-gal and displayed a phenotype identical to cells with STFs that were not treated with doxycyline. Cells exposed to doxycycline develop a large, flattened appearance, and display SA-β-gal activity; all characteristics associated with the senescence phenotype [8].

### Cell Lines

In one embodiment, the human feeder layer cells provided by this invention permit cell division to be arrested by conditionally expressing known blockers of the cell cycle. The stable introduction of full-length coding regions of cell cycle inhibitor genes or fragments of such genes under control of inducible promoters not only stops cell division, but also induces differentiation to a senescence-like state. Senescence is characterized by an increase in cell volume, a flattened morphology, and increased protein synthesis. Such cells have longer lifespan and are also substantially more resistant to environmental stresses, such as lowered pH, loss of serum factors, osmotic changes and other impedance that triggers cell death in proliferating populations. Induction of senescence increases cell stability, and allows higher concentration of secreted products, such as dopamine from black cells of the substantia nigra. The materials embodied in the invention described herein are human feeder layer cells that have been engineered such that expression of the cell cycle inhibitor switches the cells from a replicative to a protein producing premature senescent state (termed “RP shift”).

These human feeder layer cells capable of supporting growth of human stem cells, especially embryonic stem cells, that maintain a euploid chromosomal complement, do not spontaneously differentiate in vitro, have unlimited proliferative capacity, and capable of differentiating into each of the three embryonic cell layers (ectoderm, mesoderm and endoderm) are preferably used to support growth in vitro of human stem cells, most preferably embryonic stem cells, that are used in biological therapies to replace dead or dying cells and revive normal function of the surrounding tissue. In biological therapy, stem cells are introduced into the site of treatment at an early stage of differentiation. These cells migrate to multiple sites and may be converted into many types of cells by signaling factors within the target tissue. These cells are then stabilized in situ by addition of chemical inducer of STFs. These cells are resistant to cell death and stop division, thus avoiding uncontrolled proliferation of the stem cells, and triggering terminal differentiation. This combination of tissue-derived signaling factors and ongoing premature senescence will force these stem cells to differentiate into the proper cell type.

### Use of Feeder Layer Cell Lines

Human embryonic stem (hES) cells hold great promise for future use in various research areas, such as human developmental biology and cell-based therapies. Traditionally, these cells have been cultured on mouse embryonic fibroblasts (or human fibroblasts cells) as a feeder layer, which permit continuous growth in an undifferentiated stage. To use these unique cells in human therapy, an animal-free culture system must be used, which will prevent exposure to mouse retroviruses. Animal-free culture systems for hES cells enjoy three major advantages in the basic culture conditions: 1) the ability to grow these cells under serum-free conditions, 2) maintenance of the cells in an undifferentiated state on Matrigel matrix with 100% human fibroblasts cells-conditioned medium, and 3) the use of either human embryonic fibroblasts or adult fallopian tube epithelial cells as feeder layers. Here are described human feeder layer cells capable of supporting growth of human stem cells, especially embryonic stem cells, that maintain a euploid chromosomal complement, do not spontaneously differentiate in vitro, have unlimited proliferative capacity, and capable of differentiating into each of the three embryonic cell layers (ectoderm, mesoderm and endoderm).

Several normal human cells, such as hematopoietic stem cells, dendritic cells, and B cells, can be cultured in vitro in defined optimal conditions. Several ex vivo culture systems require the use of feeder cells to support the growth of target cells. In such systems, proliferation of feeder cells has to be stopped, so that they can be used as nonreplicating viable support cells. Because feeder cells need to provide one or few active signals, it is important to maintain them in a metabolically active state, allowing continued expression of specific ligands or cytokines. Mitomycin C and gamma-irradiation treatments are commonly used to prepare nonproliferating feeder cells and are usually considered to be equivalent. Normal human B lymphocytes can be expanded in vitro in the presence of feeder cells expressing the CD40 ligand CD154. Provided herein are human feeder layer cells capable of supporting growth of human stem cells, especially embryonic stem cells, that maintain a euploid chromosomal complement, do not spontaneously differentiate in vitro, have unlimited proliferative capacity, and capable of differentiating into each of the three embryonic cell layers (ectoderm, mesoderm and endoderm) alternative to gamma-irradiation- and mitomycin C-treated feeder cells. There is a significant reduction in both cellular metabolism and level of CD154 expression observed in mitomycin C-treated feeder cells. These results indicate that mitomycin C-treated feeder cells are metabolically altered, and consequently less efficient at maintaining cell expansion in the long-term cell culture system used.

RP Shift methods as disclosed herein for stem cell lines provide an alternative for producing senescent feeder cell layers for promoting hES cell growth.

### Preparation of RP Shift in Stem Cell Feeder Cell Lines

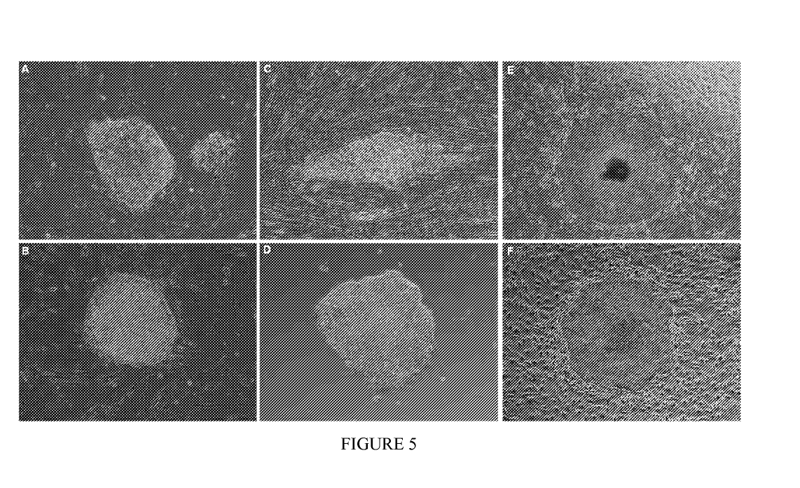

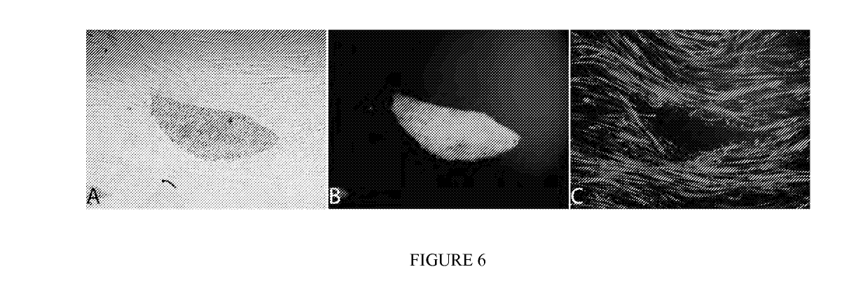

Previously, other groups have demonstrated that human cells such as foreskin fibroblasts, and placental fibroblast can support the long term growth of hESCs [Amit et al., 2004, Feeder layer-and serum-free culture of human embryonic stem cells. Biol Reprod, 70(3): p. 837-45; Beattie et al., 2005, Activin A maintains pluripotency of human embryonic stem cells in the absence of feeder layers. Stem Cells, 23(4): p. 489-95; Xu et al., 2005, Basic fibroblast growth factor supports undifferentiated human embryonic stem cell growth without conditioned medium. Stem Cells, 23(3): p. 315-23]. Several human foreskin fibroblast lines were generated using standard procedures, and one line transformed using lentivirus (as set forth in Mitta et al., 2002, Advanced modular self-inactivating lentiviral expression vectors for multigene interventions in mammalian cells and in vivo transduction. Nucleic Acids Res, 30(21): p. e113) bearing red fluorescent monomer (Campbell et al., 2002, A monomeric red fluorescent protein. Proc Natl Acad Sci USA, 99(12): p. 7877-82) and hTERT (Counter et al., 1998, Telomerase activity is restored in human cells by ectopic expression of hTERT (hEST2), the catalytic subunit of telomerase. Oncogene, 16(9): p. 1217-22). hESCs were viable and expanded as expected on normal and transformed human foreskin feeders (FIG. 5C, FIG. 6). These experiments were conducted using the highest quality ES qualified reagents and similar media components to derive all human feeder layers. However, most lines were generated using fetal bovine serum. In addition to primary and transformed lines, which still require mitotic inactivation (irradiation) prior to feeder layer preparation, a novel human fibrosarcoma feeder cell line was produced (shown in FIG. 5, Panels E and F). This line has been genetically modified to contain RP Shift expression vector, an IPTG inducible p16/p21 gene cassette that causes premature cell senescence and anti-apoptotic cell protection. Addition of IPTG to these cells arrests fibrosarcoma proliferation, doesn't affect the ability of hESCs to divide and maintain apparent pluripotency (as indicated by Oct4 promoter activity, data not shown), and provides apparent adequate support to expand all of the above mentioned hESCs.

Results. RP Shift competent human fibroblast feeder layer cells were plated onto P50 dishes coated with gelatin that were seeded on day 0 with 100,000 RP Shifted cells. After 24 hours, 10 uM IPTG was added to the culture to trigger the RP Shift. Control cultures without IPTG (data not shown) and cells that were irradiated instead with (80 Gy) gamma irradiation. H7 or BG01 cells (100,000) were plated on the different feeder layers in separate cultures and allowed to form colonies over 10 days. The number of colonies and their size were determined using a Nikon microscope with 10× and 20× objective respectively. Two to three times the number of colonies with approximately 2-4 times average diameter were measured for both ES cell lines that were grown on RP Shifted feeder layers than on irradiated feeder layers. In total, four to six times the number of ES cells were grown on RP Shifted feeder layer cells than on comparable irradiated feeder layers.

Stem cell lines are commercially available embryonic stem cell lines (TABLE 3):

TABLE 3
Commercially available embryonic stem cell lines

| Cell line | Source |
|---|---|
| BG01, BG02, BG03 | BresaGen, Inc. |
| SA01, SA02 | Cellartis |
| ES01, ES02, ES03, ES04, | ES Cell International |
| ES05, ES06 | |
| MI01 | MizMedi Hospitals |
| TE03, TE04, TE06 | Technicon Israeli Institute |
| | of Technology |
| UC06 | University of California |
| | San Francisco |
| WA01, WA07, WA09, | WiCell Research Institute |
| WA13, WA14 | |

In one embodiment, the feeder layer cell line contains an expression vector encoding a Cip/Kip protein family member, or INK4A, or one or a plurality of Cy protein motifs or one or a plurality of ankyrin-binding protein motifs, or a combination of one or a plurality of Cy protein motifs and one or a plurality of ankyrin-binding protein motifs. In the latter embodiments the one or a plurality of Cy protein motifs and one or a plurality of ankyrin-binding protein motifs can be encoded in one or more than one expression vector.

The expression vector can, for example, be in the form of a plasmid, cosmid, viral particle, or phage. The appropriate nucleic acid sequence can be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, preferably a selectable marker gene, an enhancer element, a promoter, and a transcription termination sequence. Construction of suitable vectors containing one or more of these components employs standard ligation techniques that are known to the skilled artisan.

Expression vectors may contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. However, in mammalian systems, it is preferred that the vector integrate into the genome thereby becoming dependent on the host for replication. Thus, in certain embodiments, the vector is a retrovirus-based vector.

Expression vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Expression vectors usually contain a promoter operably linked to the polypeptide-encoding nucleic acid sequence to direct mRNA synthesis. Promoters recognized by a variety of potential host cells are well known. Promoters suitable for use with prokaryotic hosts include the β-lactamase and lactose promoter systems, alkaline phosphatase, a tryptophan (trp) promoter system, and hybrid promoters such as the tac promoter. Promoters for use in bacterial systems also will contain a Shine-Dalgarno (S.D.) sequence operably linked to the polypeptide encoding region.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription in higher eukaryotes may be increased by inserting an enhancer or repressor sequence into the vector. Enhancers and repressors are cis-acting elements of DNA, usually about from 10 to 300 bp that act on a promoter to increase its transcriptional activity. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers.

In one embodiment, an expression vector of the present invention contains a senescence-responsive element to increase the production of a recombinant protein. Additional amplification of the desired recombinant protein is achieved by engineering a senescence-responsive element into the vector upstream of a viral promoter, such as the CMV promoter. A senescence-responsive element has been defined at the −89 to −66 sequence (5′-AGGATGTTATAAAGCATGAGTCA-3′; SEQ ID NO:2) of the human collagenase gene. Development of the senescence phenotype by expression of the STFs activates senescence-specific transcription factors, thereby accelerating transcription of the recombinant protein of interest. In certain embodiments, the senescence-responsive element may be operably connected to a bicistronic construct comprising a combination of desired recombinant product and the IRES-driven cell cycle inhibitors separately or both transcribed from the regulated promoter. Such a dicistronic design provides simultaneous regulated expression of the target protein and the cell cycle regulator.

In certain embodiments, an expression vector of the present invention contains elements that allow tight regulation of gene expression. For example, the expression vector may contain one or more tetracycline repressor binding sites (tetracycline operators) in the promoter region of the vector. In one embodiment, the vector comprises multiple tetracycline operators and a minimal promoter comprising a TATA sequence. Preferably, the tetracycline operators are arranged to provide tight regulation of the promoter. One such arrangement includes two phased tetracycline operators 21 basepairs downstream from the TATA sequence and two phased tetracycline operators 11 basepairs upstream from the TATA sequence.

When vectors comprising tetracycline repressor binding regions are used, it is necessary to deliver the tetracycline repressor into the cells chosen for biopharmaceutical production. The tetracycline repressor may be introduced into these producer cells via a retroviral transduction using IRES-containing single-transcript vector (Levenson et al., 1998, Hum Gene Ther. 9: 1233-6). After these producer cells are modified to express tetracycline repressor, the tetracycline-regulated construct containing the CKI is integrated into the genome of the producer cells by retroviral infection. Cells harboring the RP shift vector as stable transductants may be selected by resistance to the antibiotic G418. The expression of the delivered CKI or other cell-cycle inhibitor is then induced by adding doxycycline (a stable derivative of tetracycline) into the media. Expression using tetracycline-responsive promoters is known in the art, for example as disclosed in U.S. Pat. No. 5,804,604.

Expression vectors used in eukaryotic host cells (yeast, fungi, insect, plant, animal, human, or nucleated cells from other multicellular organisms) can advantageously also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polypeptide.

The RP shift vector is described in U.S. Pat. No. 6,635,448 and U.S. Patent Application Publication No. US-2006-0051861-A1, the contents of each of which are fully incorporated for all purposes by this reference. The expression vector, must be (1) tightly regulated, to allow robust cell growth when in the OFF position; (2) highly inducible by inexpensive and FDA-approved ligand; and (3) very promiscuous to allow efficient incorporation and subsequent expression in a wide variety of cells. These requirements are fulfilled by the disclosed tetracycline-regulated retroviral vector. Although the vector was built from a commercially-available plasmid (T-REx plasmid vector from Invitrogen), several modifications have been made to adapt the vector to the purposes of the invention. In its final form, the vector is arranged to express a selection marker from viral long terminal repeat (LTR) sequence, and further comprises a multiple cloning site flanked by a polyadenylation signal and a regulated promoter.

cDNAs for the CKIs, in sense orientation, or other inhibitors of the cell cycle (see following sections), are cloned into the multiple cloning site (MCS) to be expressed from the regulated promoter. The regulated promoter and polyadenylation site are positioned in reverse orientation to the LTR to prevent read-through from the LTR, thereby eliminating LTR-initiated expression leakage. At the same time reverse orientation of the polyadenylation signal does not interfere with genomic RNA transcription in packaging cells.

The vector has been modified in particular by the insertion of two phased tetracycline operators 21 bp downstream from the TATA site and two phased tetracycline operators 11 bp upstream of the TATA sequence within the CMV promoter. This configuration positions a tight protein clamp of two dimerized tet-repressors both in front of the TF-IID contact site and also exactly at the site of initiation of transcription. Moreover, binding of dimerized tetracycline repressors induces a significant kink in the double helix, further reducing the probability of fortuitous transcription. Thus the vector is particularly adapted to prevent expression of any cloned sequence in the absence of an inducing signal recognized by the cognate repressor protein.

The second component of the system is the tetracycline repressor, modified to incorporate a nuclear localization signal. Triggering the senescence phenotype by expression of the STFs should activate as yet undetermined senescence-specific transcription factors, thereby accelerating transcription of the recombinant protein of interest. Experiments testing several modifications of the regulated cassette, including a combination of desired recombinant product and the IRES-driven cell cycle inhibitors separately or both transcribed from the regulated promoter. Such a dicistronic design provides simultaneous regulated expression of the target protein and the cell cycle regulator.

### Preparation of Stem Cell Feeder Layer Cells Containing the RP Shift Construct

Human ES cell lines have been derived from the inner cell mass of preimplantation embryos by culturing the cells on mouse embryonic feeder cells [3, 27]. Under these conditions, human ES cells show remarkable proliferative capacity and stability in long-term culture [28] and have the capacity to differentiate into cell types from all three germ layers both in vitro and in vivo [3, 27]. Therefore, human ES cells may be a source of cells for cell therapies, drug screening, and functional genomics applications. These cells can be maintained in long-term culture on feeders. As disclosed herein, methods were developed for maintaining the hES cells using feeder cells undergoing RP Shift as well as standard irradiation protocols. In addition, methods were developed for culturing hES cells in feeder-free systems. Using these methods, cells cultured under these conditions maintain a normal karyotype and a stable proliferation rate, express SSEA-4, Tra-1-61, Tra-1-80, alkaline phosphatase, hTERT and OCT-4 and have the capacity to differentiate into cell types from the three germ layers both in vitro and in vivo [29]. In view of the particularity with which stem cells must be cultured, set forth as follows are detailed protocols for maintaining hESCs using feeder layer cells.

1. Materials

1.1 Solutions and media

1.1.1 Stocks
1. Collagenase IV solution (200 units/mL). Dissolve 20,000 units of collagenase IV (GibcoBRL/Invitrogen cat# 17104-019) in 100 mL Knockout □ DMEM. Add all components to a 250 mL filter unit (0.22 μM, Corning, cellulose acetate, low protein-binding) and filter. Aliquot and store at −20° C. until use. Note: In our hands, 200 units/mL is usually 1 mg/mL.
2. DMEM, high-glucose, without glutamine (GibcoBRL/Invitrogen cat# 11965-092).
3. Fetal bovine serum (FBS) (Hyclone cat#SH30071-03).
4. Gelatin (0.5%). Add 100 mL 2% gelatin (Sigma cat# G1393) and 300 mL water for embryo transfer (Sigma cat# W1503) into a 500 mL filter unit (0.22 μM, Corning, cellulose acetate, low protein-binding) and filter. Store at 4° C.
5. L-glutamine solution (200 mM). (GibcoBRL/Invitrogen cat# 25030-081) Make aliquots of 10 mL and store at −20° C.
6. Human basic fibroblast growth factor, recombinant (hbFGF) (10 μg/mL). Dissolve 10 μg hbFGF (GibcoBRL/Invitrogen cat #13256-029) in 1 mL PBS with 0.2% BSA (Fraction V, Sigma cat #A9576). Filter the solution using a 0.22 μM, Corning, cellulose acetate, low protein-binding filter. When handling hbFGF, pre-wet all pipet tips, tubes and the filter with PBS+0.2% BSA. hbFGF is very sticky and this will prevent some loss of the bFGF. Store stock at −20° C. or −80° C. (for long-term storage, keep stocks at −80° C.). Store thawed aliquots at 4° C. for up to 1 month.
7. Knockout□ DMEM (GibcoBRL/Invitrogen cat# 10829-018).
8. Knockout□ Serum Replacement (GibcoBRL/Invitrogen cat #10828-028).
9. Matrigel®. Either growth factor-reduced Matrigel® (Becton Dickinson cat# 356231) or regular Matrigel® (Becton Dickinson cat# 354234) can be used for coating plates. To prepare Matrigel® aliquots, slowly thaw Matrigel® at 40 C overnight to avoid the formation of a gel. Add 10 mL of cold Knockout□ DMEM to the bottle containing 10 mL Matrigel®. Keeping the mixture on ice, mix well with a pipet. Aliquot 1-2 mL into each pre-chilled tube; store at −200 C.
10. β-mercaptoethanol (1.43M) 14.3M β-mercaptoethanol (Sigma cat# M 7522) is diluted 1:10 in PBS and stored at −20° C. or −80° C. in 40 μL aliquots. Aliquots are thawed and used immediately; do not re-use.
11. Non-essential Amino Acids (10 mM) (GibcoBRL/Invitrogen cat# 11140-050).
12. D-PBS without Ca2+Mg2+ (GibcoBRL/Invitrogen cat# 14190-144).
13. Trypsin/EDTA (0.05% trypsin, 0.53 mM EDTA) (GibcoBRL/Invitrogen cat# 25300-054).

1.1.2 Media
1. Human fibroblasts cell medium. Add all medium components listed below to 500 mL filter unit (0.22 μM, Corning, cellulose acetate, low protein-binding) and filter. Store at 4° C. and use within 1 month.
   a. 450 mL DMEM (GibcoBRL/Invitrogen cat# 11965-092)
   b. 50 mL FBS (final concentration 10%, heat inactivated, Hyclone cat# 30071-03)
   c. 5 mL 200 mM L-glutamine (final concentration 2 mM)
2. Human ES medium. Add all medium components listed below to 500 mL filter unit (0.22 μM, Corning, cellulose acetate, low protein-binding) and filter. Store at 4° C. for no longer than 2 weeks.
   a. 400 mL Knockout □ DMEM (GibcoBRL/Invitrogen cat #10829-018)
   b. 100 mL Knockout □ Serum Replacement (GibcoBRL/Invitrogen cat #10828-028),
   c. 5 mL Non-essential Amino Acids (GibcoBRL/Invitrogen cat #11140-050)
   d. 2.5 mL 200 mM L-glutamine (final concentration of 1 mM)
   e. 35 μl 0.14 M β-mercaptoethanol (Sigma Cat#M7522) (final concentration of 0.1 mM)
3. Differentiation medium. The differentiation medium is made by replacing Knockout C Serum Replacement with 20% FBS (not heat inactivated) in the human ES medium described above.
4. Cryopreservation medium. Add all medium components listed below to 100 mL filter unit (0.22 μM, Corning, cellulose acetate, low protein-binding) and filter.
   a. 60 mL Knockout□ DMEM (for ES cells) or 60 mL DMEM (for human fibroblasts)
   . 20 mL FBS
   c. 20 mL DMSO (Sigma cat# D2650) without filtration.

1.2. Tissue culture plates and flasks 6-well plates (Falcon cat# 3046) are used for ES culture; T75 flask (Corning cat# 430641), T150 flask (Corning cat# 430825) and T225 flask (Corning cat# 431082) for human fibroblasts cell culture; 6-well low attachment plates (Corning, cat# 29443-030) for embryoid bodies; 4-well plates for thawing human ES cells (Nunc #176740).

1.3. Incubators All cells are maintained under sterile conditions in a humidified incubator in a 5% CO₂-95% air atmosphere at 370 C.

2. Methods

2.1. Human Fibroblasts Cell Manipulation

2.1.1 Cryopreservation of Human Fibroblasts Cells
1. Pre-label all cryovials with the following information: cell line, passage number, number or surface area of cells frozen, date, and initials.
2. Aspirate human fibroblasts cell medium from flask.
3. Wash cells once with PBS without Ca2+Mg2+ (2-3 mL/T75 and 5-10 mL/T150).
4. Add trypsin/EDTA to cells (1.0 mL/T75 and 1.5 mL-2.0/T150), and rock flask back and forth to evenly distribute the solution. Incubate for approximately 5 minutes at 37° C.
5. Detach cells from the plate by pipetting off or tapping the flask against the heel of your hand.
6. Neutralize trypsin/EDTA with human fibroblasts cell medium (5 mL/T75, 10 mL/T150).
7. Pipet to break up clumps of cells. If clumps remain, add suspension to a 50 mL tube and allow the chunks to settle out.
8. Perform cell count of the cell suspension to determine the number of vials you will be freezing down. 10-20 million cells per 1 mL are typically frozen in each vial.
9. Pellet the cells by centrifugation for 5 min at 300×g.
10. Resuspend pellet in 0.5 mL of DMEM with 20% FBS per vial (This is one half the final volume required for freezing).
11. Dropwise, add an equivalent volume (0.5 mL per vial) of Cryopreservation Medium and mix. The DMSO concentration is now 10%.
12. Place 1 mL of cell mixture into each freezing vial.
13. Rapidly transfer the cells to a Nalgene freezing container (Fisher cat# 15-350-50) and place at −70° C. overnight. Transfer cells to liquid nitrogen the next day for long-term storage. Alternatively, cells can be frozen using a controlled-rate freezer and transferred to liquid nitrogen at the completion of the freeze cycle.

2.1.3 Thawing and maintaining human fibroblasts cells
1. Remove vial from liquid nitrogen.
2. Do a quick thaw in a 37° C. water bath. Carefully swirl vial in 37° C. water bath, being careful not to immerse the vial above the level of the cap.
3. When just a small crystal of ice remains, sterilize the outside of the vial with 95% EtOH. Allow EtOH to evaporate before opening the vial.
4. Gently pipet the cell suspension up and down once, and place it into a 15 mL conical tube.
5. Add 10 mL warm human fibroblast medium to the tube dropwise to reduce osmotic shock.
6. Centrifuge the cell suspension at 300×g for 5 minutes.
7. Remove the supernatant.
8. Resuspend the cell pellet in 10 mL (T75) or 20 mL (T150) culture medium, and add to the flask. Plating density will need to be determined for each lot. About 5×10⁴ to 1.5×10⁵ cells/cm² are used.
9. Place in 37° C. incubator.
10. Replace the medium the next day and split the human fibroblasts 2 to 3 days after thawing, when they become 80%-85% confluent.
11. The human fibroblasts are split 1:2 every other day with trypsin, keeping cells subconfluent. human fibroblasts are only used through passage 5.

2.1.4 Irradiating & Plating human fibroblasts
1. Coat flasks or plates with gelatin by adding 0.5% gelatin at 15 mL/T225, 10 mL/T150, 5 mL/T75 and 1 mL/well of 6-well plates and incubate at 37° C. for at least 1 h. Use flasks or plates 1 h to 1 day after coating. Remove gelatin solution immediately before use.
2. Aspirate medium from human fibroblasts culture.
3. Wash cells once with PBS without Ca2+/Mg2+ (2-3 mL/T75, 5-10 mL/T150).
4. Add trypsin/EDTA (1.0 mL per T75 and 1.5 mL per T150), and incubate at 370 C until cells are rounded up (usually 3-10 mins). To loosen cells, either tap flask against the heel of your hand or pipet them off.
5. Add 9 mL human fibroblast medium, collect cells in a 15 mL conical tube and pipet several times to dissociate the cells. Perform a cell count by mixing cell suspension thoroughly and removing 10 μl. Add this to 10 μl Trypan Blue and mix well. Add ≦10 μl cell suspension/Trypan Blue mix to hemacytometer. Count the phase bright cells.
6. Irradiate cells at 40-80 Gy. This number is variable between fibroblast sources—the goal is to irradiate them enough to stop them from growing, but not enough to kill them.
7. Spin down cells at 300×g for 5 min and discard supernatant.
8. Resuspend cells in appropriate volume of human fibroblast medium. Plate at 56,000 cells/cm2 for cultures to be used for CM. The final volumes should be 3 mL/well for a 6 well plate, 10 mL/T75, 20 mL/Ti50 and 50 mL/T225.
9. When placing the plate in the incubator, gently shake the plate left to right and back to front to obtain an even distribution of cells.
10. Irradiated human fibroblasts can be used up to 7 days for CM.

2.2 Culture of Human ES Cells with Matrigel® and Conditioned Medium

2.2.1 Preparation of Conditioned Medium (CM)

A schematic diagram of this culture system is shown in FIG. 1.
1. Plate irradiated human fibroblasts cells at 56,000 cells/cm2 in human fibroblast medium as described in section 2.1.4. The final volumes should be 3 mL/well for a 6 well plate, 10 mL/T75, 20 mL/T150 and 50 mL/T225.
2. To condition medium, replace human fibroblast medium with human ES medium (0.5 mL/cm2) supplemented with 4 ng/mL hbFGF (0.4 mL/cm2) one day before use.
3. Collect CM from feeder flasks or plates after overnight incubation, and add an additional 8 ng/mL hbFGF.
4. Add fresh human ES serum replacement medium containing 4 ng/mL hbFGF (0.4 mL/cm2) to the feeders.
5. The human fibroblasts cells can be used for 1 week, with CM collection once every day.

2.2.2 Preparation of Matrigel®-coated plates
1. Slowly thaw Matrigel® aliquots at 40 C for at least 2 h to avoid the formation of a gel.
2. Dilute the Matrigel® aliquots 1:15 in cold Knockout FDMEM (for a final dilution of 1:30).
3. Add 1 mL of Matrigel® solution to coat each well of a 6-well plate.
4. Incubate the plates 1-2 h at RT, or at least overnight at 40 C. The plates with Matrigel® solution can be stored at 40 C for one week.
5. Remove Matrigel® solution immediately before use.

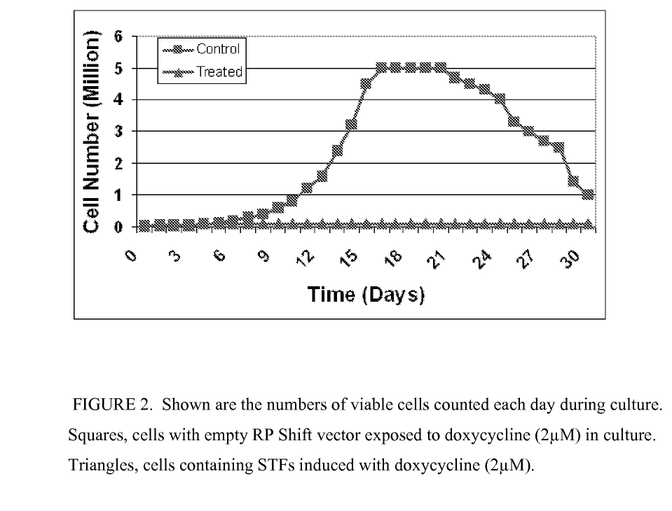

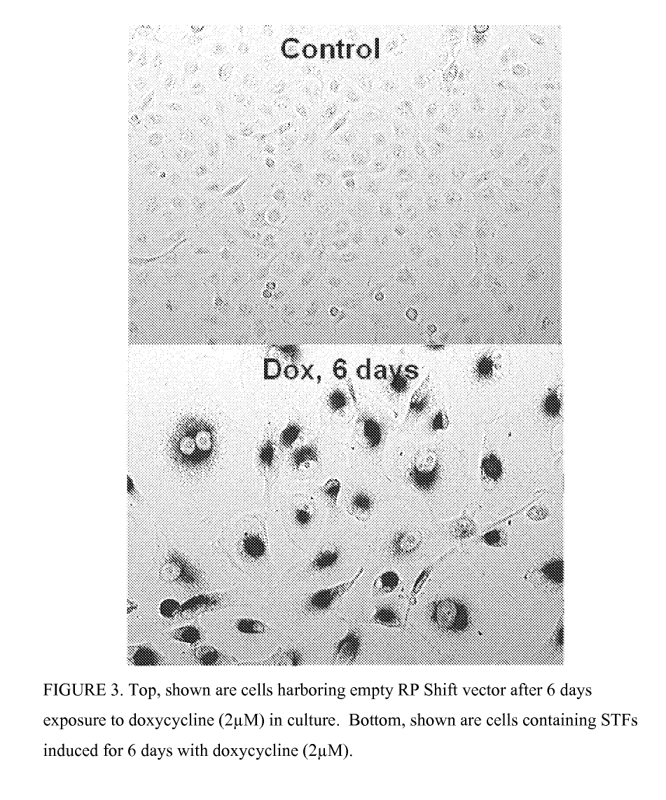

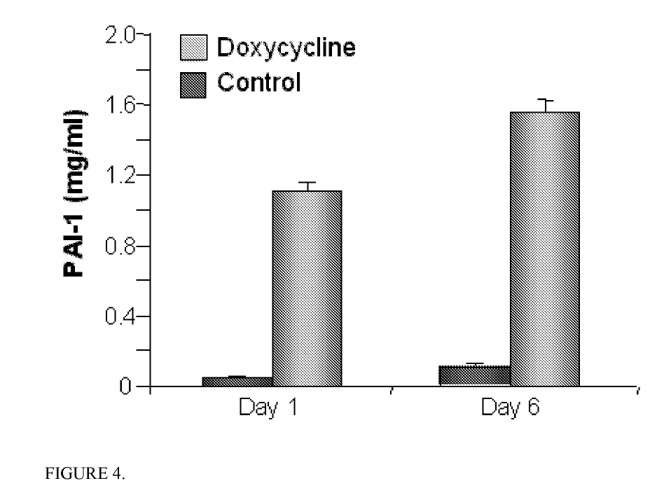

2.2.3 Passage of human ES cells on Matrigel® Phase images of typical hES cell cultures grown in feeder-free conditions are show in FIG. 2.
1. Aspirate medium from human ES cells, and add 1 mL of 200 units/mL collagenase IV per well of 6-well plate.
2. Incubate 5-10 minutes at 37° C. in incubator. Incubation time will vary among different batches of collagenase; therefore, determine the appropriate incubation time by examining the colonies. Stop incubation when the edges of the colonies start to pull away from the plate.
3. Aspirate the collagenase, and gently wash once with 2 mL PBS.
4. Add 2 mL of CM into each well.
5. Gently scrape cells with a cell scraper or a 10-mL pipet to collect most of the cells from the well, and transfer cells into a 15-mL tube.
6. Gently dissociate cells into small clusters (50-500 cells) by gently pipetting. Do not triturate cells to a single cell suspension.
7. Remove Matrigel®-containing solution from the plates.
8. Seed the cells into each well of Matrigel®-coated plates. The final volume of medium should be 4 mL per well. In this system, the human ES cells are maintained at high density. At confluence (usually one week in culture) the cells will be at 300,000-500,000 cells/cm2. An optimal split ratio is 1:3 to 1:6. Using these ratios, the seeding density is approx 50,000-150,000 cells/cm².
9. Return the plate to the incubator. Be sure to gently shake the plate left to right and back to front to obtain even distribution of cells (do not swirl the dish as the cells will collect in the middle of the dish).
10. The day after seeding, undifferentiated cells are visible as small colonies. Single cells in between the colonies will begin to differentiate. As the cells proliferate, the colonies will become large and compact, representing the majority of surface area of the culture dish (FIG. 2).

2.2.4. Daily maintenance of feeder-free culture
1. Collect CM from feeders, filter using a 0.2 μm filter, and add hbFGF to a final concentration of 8 ng/mL.
2. Feed human ES cells with 4 mL CM supplemented with hbFGF for each well of 6-well plates every day.
3. Passage when cells are 100% confluent. At this time, the undifferentiated cells should represent at least 80% of the surface area. The cells in between the colonies of undifferentiated cells appear to be stroma-like cells. The colonies (but not the stroma-like cells) show positive immunoreactivity for SSEA-4, Tra-1-60, Tra-1-81 and alkaline phosphatase.

2.2.5. Notes on feeder-free culture
1. When feeding cells, only prepare the amount of medium needed each time. For each aliquot of medium, add the appropriate amount of hbFGF (4-8 ng/mL). Place this aliquot in a 370 C water bath until warm. Use immediately. Do not heat up the entire bottle of medium, as the Knockout□0 DMEM and Knockout□ Serum Replacement do not tolerate repeated warming and cooling.
2. The CM can be filtered using a 500 mL filter unit (0.22 μM, Corning, cellulose acetate, low protein-binding) and stored at −200 C before use.
3. Other matrix proteins also support human ES growth in human fibroblasts cells -CM. Cells can be maintained on laminin, fibronectin or collagen IV-coated plates. Cultures on laminin contained a high proportion of undifferentiated colonies, which continued to display ES cell-like morphology after long-term passage. However, cells maintained on fibronectin or collagen IV had fewer colonies that displayed appropriate undifferentiated ES-morphology.
4. Cell lines such as STO (immortal mouse embryonic fibroblast cell line) and NHG190 (triple drug resistant, GFP-positive mouse embryonic cell line transfected with hTERT, unpublished data) can produce effective CM. Routine culture medium for STO cells is human fibroblast medium supplemented with 0.1 mM non-essential amino acids. NHG190 medium is human fibroblast medium supplemented with an additional 10% FBS. Cells can be harvested and irradiated at 40 Gy, counted, and seeded at about 38,000/cm2 for NHG190 cells or 95,000/cm2 for STO cells. After at least 4 h, exchange the medium with human ES medium (0.5 mL/cm2) for production of CM.

2.3 Freezing Human ES Cells
1. Treat cells with 200 U/mL collagenase IV for approximately 5-10 minutes at 37° C. (until edges of colonies are curling up). Remove collagenase and add 2 mL human ES medium per well.
2. With a 5 mL pipet, gently pipet and scrape colonies from plate. Add cell suspension to a 15 mL centrifuge tube and GENTLY break up colonies. It is important to be gentle in this step as “chunkier” colonies will thaw better than will single cells. Ideally, colonies meant for freezing are left slightly larger than they would be for splitting.
3. Centrifuge 5 minutes at 300×g.
4. Resuspend pellet (gently) in 0.25-0.50 mL DMEM with 20% FBS per vial (This is one-half the final volume required for freezing). For feeder-free cultures, 30% Knockout□ Serum Replacement can be substituted for 20% FBS.
5. Dropwise, add an equivalent volume (0.25-0.50 mL per vial) of Cryopreservation Medium and mix. The DMSO concentration is now 10%.
6. Place 0.5-1.0 mL of cell mixture in each freezing vial.
7. Rapidly transfer the cells to a Nalgene freezing container (Fisher cat# 15-350-50), and place immediately at −70° C. overnight (do not leave cells in DMSO at room temperature for long periods of time). Transfer cells to liquid nitrogen the next day for long-term storage. Alternatively, cells can be frozen using a controlled-rate freezer and transferred to liquid nitrogen at the end of the freeze cycle.

2.4 Thawing Human ES cells
1. Remove human ES cells from the liquid nitrogen storage tank
2. Thaw cryovial by gently swirling in a 37° C. water bath until only a small ice pellet remains, being careful not to completely submerge the cryovial under water.
3. Completely submerge the cryovial in 95% ethanol. Allow the vial to air dry before opening vial.
4. Very gently, pipet cells from the vial into a 15 mL conical centrifuge tube.
5. Slowly, add 9.5 mL of medium dropwise to reduce osmotic shock. While adding medium, gently mix the cells in the tube by gently tapping the tube with a finger.
6. Centrifuge at 150×g for 5 minutes.
7. Resuspend in 0.5-1.0 mL, and add 0.5 mL per well of a 4-well plate that is already coated with Matrigel®.
8. Change medium daily; however, it may take 2 weeks before cells are ready to be expanded.

2.5 Formation of Embryoid Bodies In vitro differentiation can be induced by culturing the human ES cells in suspension to form embryoid bodies. Differentiation of human ES into neurons, cardiomyocytes, and endoderm cells has been induced using the following procedure. 1. Aspirate medium from human ES cells, and add 1 mL/well of collagenase IV (200 u/mL) into 6-well plates. 2. Incubate 5 minutes at 37° C. in an incubator. 3. Aspirate the collagenase IV and gently wash once with 1 mL PBS. 4. Add 2 mL of differentiation medium into each well. 5. Scrape cells with a cell scraper or pipet and transfer cells to one well of a low attachment plate (1:1 split). Cells should be collected in clumps. Add 2 mL of differentiation medium to each well to give a total volume of 4 mL per well. Depending on the density of the hES cells, the split ratio for this procedure can vary. 6. After overnight culture in suspension, ES cells form floating aggregates known as embryoid bodies (EBs). 7. To change the medium, transfer EBs into a 15-mL tube and let aggregates settle for 5 min. Aspirate the supernatant, replace with fresh differentiation medium (4 mL/well), and transfer to low attachment 6 well plates for further culture. 8. Change medium every 2 to 3 days. During the first days, the EBs are small with irregular outlines; they increase in size by day 4. The EBs can be maintained in suspension for more than 10 days. Alternatively, EBs at different stages can be transferred to tissue culture plates for further induction of differentiation.

STFs are introduced into stem cells or EBs by infectious viral particles using an expression vector of the present invention and the subsequent infection of target cells. This delivery system employs a pantropic system to deliver the DNA to the cells. VSV-G, an envelope glycoprotein, is used to mediate viral entry into cells through lipid binding and plasma membrane fusion {Burns, 1993 #45; Yee, 1994 #46; and {Emi, 1991 #47}. Because this system does not depend on specific cell surface receptors, the pantropic system allows transduction of any mitotically active cells. Infectious pantropic retroviral particles carrying the gene of interest were transfected into GP2-293 cells using standard Ca-phosphate technique [30]. Twenty-four hours after transfection culture medium with infectious virions was collected, filtered through 0.45 micron filter to remove stray packaging cells, supplemented with Polybrene™ (4 micrograms/ml) and added to the target cells. Twenty-four hours later cells were typsinized and re-plated: two 60-mm plates will be seeded with 200 cells each, while the rest of the cells is plated into 150-mm plates at a density of 1.0sup.6 cells per plate. All cells in 150-mm plates and one of the 60-mm plates were selected with G418 (0.7 mg/ml for 10 days) to eliminate uninfected cells. The second 60-mm plate is left without antibiotic. An infection rate of 55% was determined by colony formation by dividing the number of colonies in G418-treated 60-mm plate by the number of colonies in its untreated companion.

Target cell lines must be able to encounter and receive viral particles produced by packaging cell lines. In the case of human cells, VSV-G viral coat proteins are used so that fusion of the viral particles to plasma membrane affords infection of target cells.

Samples of the G418-selected stem cell lines are tested by triggering senescence-associated beta galactosidase activity[26] by adding 2 μM of the inducer doxycyline.

The Examples which follow are illustrative of specific embodiments of the invention, and various uses thereof. They set forth for explanatory purposes only, and are not to be taken as limiting the invention.

### EXAMPLES

To demonstrate the use of the RP Shift system for producing the senescence phenotype in human feeder layer cells, the p16 and p21 elements RP Shift elements were introduced into HT420 fibrosarcoma cells. HT420 cells are an immortalized cancer cell line that shares most of the phenotypic properties of human fibroblasts. These cells were grown in Iscove's modified Eagle's medium. An IRES-containing retroviral construct was used to deliver a construct encoding bacterial lac repressor, engineered to include a nuclear localization signal. This repressor was required to prevent expression of the senescence-triggering factors in the absence of IPTG inducer during growth and preparation of the cell lines. After verification of tetracycline repressor expression by RT-PCR, each of the senescence-triggering factors p16 and p21 were introduced via the regulated RP Shift expression vector separately and in combination into the HT420 cells by retroviral infection. Cells harboring the desired senescence-triggering factor construct(s) were selected with G418. Cells were allowed to grow to near confluence in T25 flasks, and then 4 μM IPTG was added to the culture. Expression of the senescence-triggering factors prevented cell proliferation and induced a premature senescent state as evidenced by increased SA-β-Gal activity, inhibited cell division and expanded protein synthesis capacity of the cells.

### Example 1

### hESC Line Growth on Human Feeder Cell Layers

The hESC lines H1, H3, BG01V-Oct4-promoter-EGFP, and parent BG01V were expanded. The H1 line is a normal hESC, while BG01V-Oct4-promoter-EGFP line (available from Invitrogen, Inc., Carlsbaad, Calif.) affords a rapid promoter based method to visualize pluripotency. This line, as the normal BG01V (31), have abnormal karyotype, however as robust growing cell lines are excellent teaching and screening tool, especially when combined with red fluorescent feeder layers. (See FIG. 5). A master bank of these above mentioned cell lines was created, using standard techniques with KSR based medium (Knockout™ serum replacer, Invitrogen, Inc.) with MEFs (FIG. 5, Panels A and B) derived from CF-1 mice (obtained from WiCell, Madison, Wis.). Following cryopreservation and validation of frozen vials, the lines were adapted to human feeder layers, such as those derived from foreskin (FIG. 5, Panel C), HT420 fibrosarcoma (FIG. 5, Panels E and F), and on Matrigel (FIG. 5, Panel D).

Human Feeder Layers. Previously, human cells such as foreskin fibroblasts, and placental fibroblasts have been used to support long term growth of hESCs (32-34). Several human foreskin fibroblast lines were prepared using conventional methods and one such cell line was transformed using lentivirus (35) bearing red fluorescent monomer (36) and hTERT (37). hESCs grown on this line as a feeder layer were viable, and expand as expected on normal and transformed human foreskin feeders (FIG. 5, Panel C and FIG. 6). In addition to primary and transformed lines, which still require mitotic inactivation (irradiation) prior to feeder layer preparation, a novel human fibrosarcoma feeder cell was prepared (shown in FIG. 5, Panels E and F). This line has been genetically modified to contain RP Shift, an IPTG inducible p16/p21 gene cassette that causes premature cell senescence and anti-apoptotic cell protection. In these genetically-engineered cells, addition of IPTG arrested fibrosarcoma proliferation, didn't affect the ability of hESCs to divide and maintain apparent pluripotency (as indicated by Oct4 promoter activity, data not shown), and provided adequate support to expand all of the above mentioned hESCs.

These results are shown in FIG. 6. Immortalized red fluorescent human foreskin feeder layers supported growth of hESCs: BG01VOct4p-EGFP hESCs (FIG. 6, Panel B) were plated on gelatin-coated dishes seeded with 40,000 irradiated NHFF240-mRFP//hTERT cells (passage 4 of Human foreskin fibroblast transduced with mRFP, hTERT, and Neo-resistance using self-inactivating lentivirus). Colonies formed classical elliptical shapes seen with human foreskin feeder layers and maintained Oct4 promoter activity.

In addition, hESCs were grown in the absence of feeder cells (feeder-free conditions) and on synthetic extracellular matrices. In particular, Matrigel, a mouse derived matrix mixture that has been qualified to support hESC growth, was used to compare the ability of the different above-mentioned hESC lines to grow thereupon and on RP-Shift transformed feeder layers seeded onto Kappa-Caregeenan (KC), an algae derived product (FMC Biopolymer). The BG01V line (as shown in FIG. 5 to be adapted to growth on RP Shift fibrosarcoma feeder cells, was used. (Indeed KC (shown in FIG. 5, Panel F) was able to support growth of BG01V hES cells in the presence of feeders.)

### Example 2

### hESC Growth in RP Shift-Transformed Human Fibroblasts

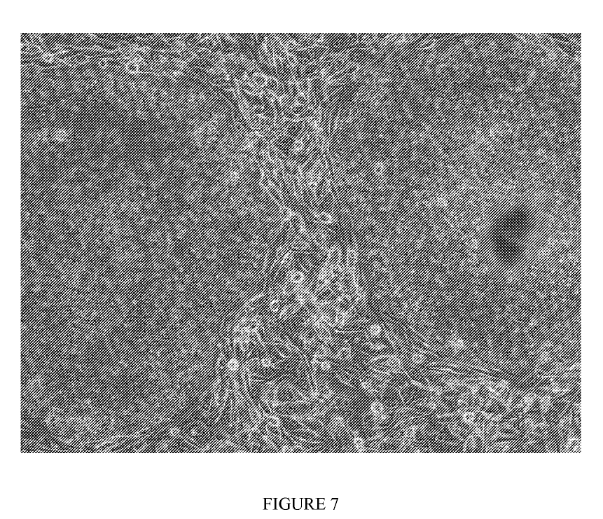

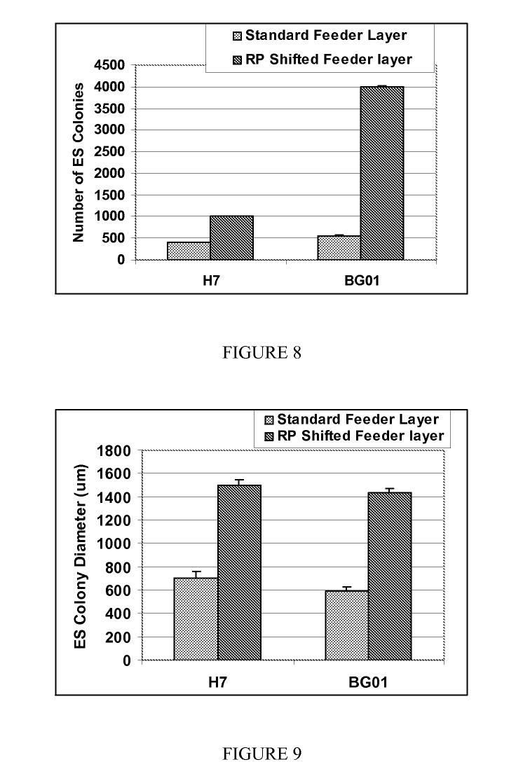

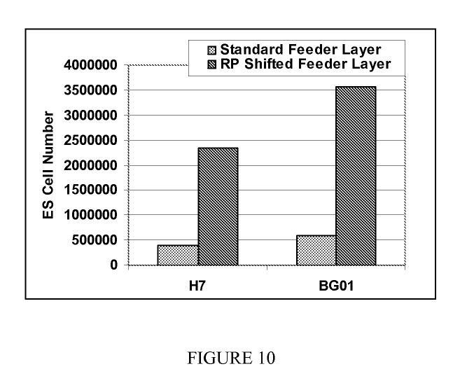

hESCs were grown on human HT1080 cells transformed with the RP Shift construct as disclosed herein; FIG. 7 illustrates hES cell growth on transformed HT1080 cells. To characterize the effectiveness of RP Shift-transformed HT1080 cells to support hESC growth, one hundred thousand hESCs (H7 or BG01) were plated on Matrigel feeder layer cells of RP Shifted HT1080 cells, or irradiated human fibroblast cells. The number of colonies was counted after 4 days; results of these experiments are shown in FIG. 8. The number of H7 colonies counted for the RP Shifted cells was more than double the number of colonies observed than normal human fibroblasts. The number of BG01 colonies found in these experiments was greater than 8-fold that of normal human fibroblasts. The size of hESC colonies was also determined by microscopic inspection using a micrometer. For both H7 and BG01 hESCs, colony size was approximately 2-fold larger for cells grown on RP Shift-transformed HT1080 cells than for cells grown on normal human fibroblast feeder cells (as shown in FIG. 9). The number of H7 and BG01 cells obtained after growth on RP Shift-transformed HT1080 cells versus normal human fibroblasts was also determined. After plating one hundred thousand hESCs on Matrigel feeder layers and growth for 4 days, the number of cells was assayed using trypan blue exclusion. For H7 hESCs, the total number of cells was more than 7-fold greater than normal human fibroblasts. The number of BG01 colonies was greater than 8-fold that of normal human fibroblasts. These results are shown in FIG. 10.

### Example 3

### hESC Growth in RP Shift-Transformed Human Fibroblast-Derived Media

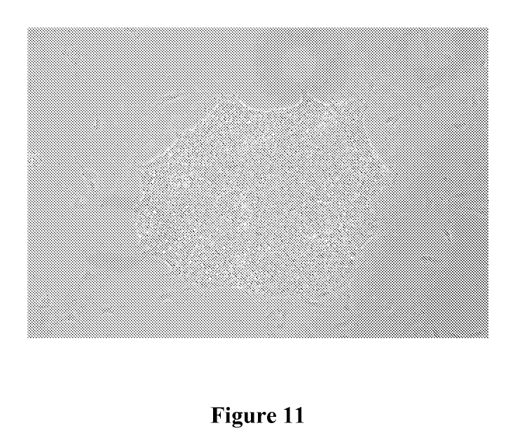

The results obtained in Example 2 suggested that soluble factor(s) secreted or otherwise produced by the RP Shifted HT1080 fibrosarcoma cells could cause the observed enhanced hESC colony and cell growth. Accordingly, hESCs were grown without feeder cells in media conditioned by RP Shift-transformed HT1080 fibrosarcoma cell growth. Media (mTeSR media) with or without addition of RP Shift-inducing amounts of IPTG was incubated with RP Shift-transformed HT1080 feeder layer cells and cultured for 2 days. The resulting conditioned media (“CmTeSR”) was added to an equal amount of fresh media (mTeSR+CmTeSR in a 50:50 ratio with IPTG) from human fibroblast cells or HT1080 fibrosarcoma cells. This mixture of conditioned medium was added to H7 cells that were grown in the absence of feeder cells (feeder free). hESC were grown under these conditions for 7 days and the number of cells in each flask was counted by trypan blue exclusion. Adding the CmTeSR media enhanced hESC H7 cell growth up to 2-fold compared to conditioned medium from irradiated human fibroblasts. H7 cells were observed microscopically to have maintained their undifferentiated state (shown in FIG. 11).

### Example 4

### Immunological Characterization of hESCs after Conditioned Media Growth

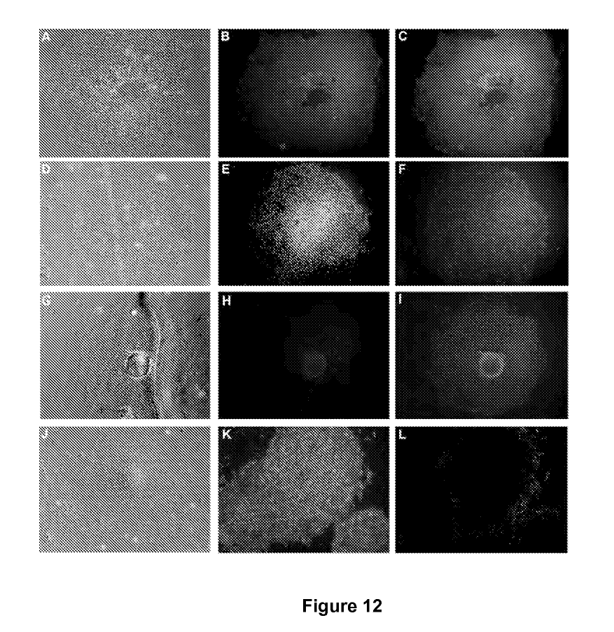

To ensure that the culturing techniques disclosed herein maintained hESC pluripotency, the immunological profile of BG01V cells plated on Matrigel was examined. BG01V hESCs were enzymatically removed from cultures containing mouse embryonic fibroblasts (MEF) feeder cells and plated onto Matrigel coated plastic coverslips (obtained from Sarstedt) in a conditioned medium formula. BG01V cells, usually grown on human feeder cell layers were seeded onto Matrigel coated surfaces in the presence of fibroblast conditioned medium as described in Example 3, and analyzed 4 days later. Initially in these experiments, hESCs accommodating to a new surface showed differentiated cells around the outer rim of the colony, whereas the center portion retained the pluripotent phenotype. These colonies were then subjected to a minimum of 3 passages with manual colony picking to launder out the spontaneously differentiated regions. The ability of the H1 and Oct4 reporter lines to grow on KC without alterations in standard hESC immunological phenotype (i.e., Oct4, Sox2, Tra markers) was also determined. These results are shown in FIG. 12.

The cultures were fixed and stained with the following combinations of antibodies (panel identification with respect to FIG. 12, where Panels A, D, G, and J are photomicrographs of colonies to show hESC morphology): SSEA4 (Panel B); Oct4 (Panel C); Sox2 (Panel E); SSEA3 (Panel F); SSEA1 (Panel H); Oct4 (Panel I); Tra-1-60 (Panel K) and Vimentin (Panel L). Under these conditions, strong SSEA4 (Panel B), Oct4 (Panels C and E), Sox2 (Panel E), SSEA3 (Panel F), and Tra-1-60 (Panel K) were observed. A small amount of SSEA1 (Panel H) was observed on a satellite colony above the main ES colony where as a minor amount of vimentin (Panel L), and nestin were observed around the rim of some colonies. However markers indicative of more advanced glial differentiation, such as GFAP, Sox 4, or Olig2, were not observed. These results indicated that hESCs could be grown in media conditioned by growth of RP Shift-transformed HT1080 cells and retain their pluripotent phenotype.

Although the present invention has been described with reference to particular embodiments, it is to be appreciated that various adaptions and modifications may be made without departing from the spirit and scope of the invention. The invention is only to be limited by the appended claims.

### REFERENCES

1. Raff, M., ADULT STEM CELL PLASTICITY: Fact or Artifact? Annual Review of Cell and Developmental Biology, 2003. 19(1): p. 1-22.
2. Wagers, A. J. and I. L. Weissman, Plasticity of adult stem cells. Cell, 2004. 116(5): p. 639-48.
3. Thomson, J. A., et al., Embryonic Stem Cell Lines Derived from Human Blastocysts. Science, 1998. 282(5391): p. 1145-1147.
4. Wichterle, H., et al., Directed differentiation of embryonic stem cells into motor neurons. Cell, 2002. 110(3): p. 385-97.
5. Isacson, O., Problems and solutions for circuits and synapses in Parkinson's disease. Neuron, 2004. 43(2): p. 165-8.
6. Kim, J. H., et al., Dopamine neurons derived from embryonic stem cells function in an animal model of Parkinson's disease. Nature, 2002. 418(6893): p. 50-6.
7. Ricordi, C. and T. B. Strom, CLINICAL ISLET TRANSPLANTATION: ADVANCES AND IMMUNOLOGICAL CHALLENGES. Nat Rev Immunol, 2004. 4(4): p. 259-268.
8. Campisi, J., Cancer, aging and cellular senescence. In Vivo, 2000. 14(1): p. 183-8.
9. Hayflick, L. and P. S. Moorhead, The serial cultivation of human diploid cell strains. Exp Cell Res, 1961. 25: p. 585-621.
10. Hayflick, L., The Limited in Vitro Lifetime of Human Diploid Cell Strains. Exp Cell Res, 1965. 37: p. 614-36.
11. Smith, J. R. and O. M. Pereira-Smith, Replicative senescence: implications for in vivo aging and tumor suppression. Science, 1996. 273(5271): p. 63-7.
12. Dimri, G. P. and J. Campisi, Altered profile of transcription factor-binding activities in senescent human fibroblasts. Exp Cell Res, 1994. 212(1): p. 132-40.
13. DAVIS, T., et al., Investigation of the Signaling Pathways Involved in the Proliferative Life Span Barriers in Werner Syndrome Fibroblasts. Ann NY Acad Sci, 2004. 1019(1): p. 274-277.
14. Hirai, H., et al., Novel INK4 proteins, p19 and p18, are specific inhibitors of the cyclin D-dependent kinases CDK4 and CDK6. Mol Cell Biol, 1995. 15(5): p. 2672-81.
15. Quelle, D. E., et al., Cloning and characterization of murine p16INK4a and p15INK4b genes. Oncogene, 1995. 11(4): p. 635-45.
16. Chang, B. D., et al., Role of p53 and p21waf1/cip1 in senescence-like terminal proliferation arrest induced in human tumor cells by chemotherapeutic drugs. Oncogene, 1999. 18(34): p. 4808-18.
17. Noda, A., et al., Cloning of senescent cell-derived inhibitors of DNA synthesis using an expression screen. Exp Cell Res, 1994. 211(1): p. 90-8.
18. Tsao, Y. P., et al., Adenovirus-mediated p21((WAF1/SDII/CIP1)) gene transfer induces apoptosis of human cervical cancer cell lines. J Virol, 1999. 73(6): p. 4983-90.
19. Flores-Rozas, H., et al., Cdk-interacting protein 1 directly binds with proliferating cell nuclear antigen and inhibits DNA replication catalyzed by the DNA polymerase delta holoenzyme. Proc Natl Acad Sci USA, 1994. 91(18): p. 8655-9.
20. Harris, W., S. Gollapudi, and S. Gupta, Anti-CD3-induced changes in protein kinase C isozymes expression in human CD4+ and CD8+ T lymphocytes. J Clin Immunol, 1995. 15(5): p. 232-41.
21. Waga, S., et al., The p21 inhibitor of cyclin-dependent kinases controls DNA replication by interaction with PCNA [see comments]. Nature, 1994. 369(6481): p. 574-8.
22. Chen, J., et al., Cyclin-binding motifs are essential for the function of p21CIP1. Mol Cell Biol, 1996. 16(9): p. 4673-82.
23. Hunt, C. R., et al., Characterization and expression of the mouse Hsc70 gene. Biochim Biophys Acta, 1999. 1444(3): p. 315-25.
24. Saha, P., et al., p21CIP1 and Cdc25A: competition between an inhibitor and an activator of cyclin-dependent kinases. Mol Cell Biol, 1997. 17(8): p. 4338-45.
25. Bucciarelli, T., Levenson, V., Primiano, T., Methods and Compositions for Increasing Protein Yield from a Cell Culture, in United States Patent and Trademark Office. 2003, Clonex Development, Inc.: United States of America.
26. Dimri, G. P., et al., A biomarker that identifies senescent human cells in culture and in aging skin in vivo. Proc Natl Acad Sci USA, 1995. 92(20): p. 9363-7.
27. Reubinoff, B. E., et al., Embryonic stem cell lines from human blastocysts: somatic differentiation in vitro. Nat Biotechnol, 2000. 18(4): p. 399-404.
28. Amit, M., et al., Clonally derived human embryonic stem cell lines maintain pluripotency and proliferative potential for prolonged periods of culture. Dev Biol, 2000. 227(2): p. 271-8.
29. Xu, C., et al., Feeder-free growth of undifferentiated human embryonic stem cells. Nat Biotechnol, 2001. 19(10): p. 971-4.
30. Pear, W. S., et al., Production of high-titer helper-free retroviruses by transient transfection. Proc Natl Acad Sci USA, 1993. 90(18): p. 8392-6.
31. Tang, X. C. and M. J. Pikal, The effect of stabilizers and denaturants on the cold denaturation temperatures of proteins and implications for freeze-drying. Pharm Res, 2005. 22(7): p. 1167-75.
32. Menendez, P., C. Bueno, and L. Wang, Human embryonic stem cells: A journey beyond cell replacement therapies. Cytotherapy, 2006. 8(6): p. 530-41.
33. Nistor, G. I., et al., Human embryonic stem cells differentiate into oligodendrocytes in high purity and myelinate after spinal cord transplantation. Glia, 2005. 49(3): p. 385-96.
34. Thomson, J. A., et al., Embryonic Stem Cell Lines Derived from Human Blastocysts. Science, 1998. 282(5391): p. 1145-1147.
35. Gibellini, D., et al., HIV-1 tat protein and cell proliferation and survival: a brief review. New Microbiol, 2005. 28(2): p. 95-109.
36. Szulc, J., et al., A versatile tool for conditional gene expression and knockdown. Nat Methods, 2006. 3(2): p. 109-16.
37. Reubinoff, B. E., et al., Embryonic stem cell lines from human blastocysts: somatic differentiation in vitro. Nat Biotechnol, 2000. 18(4): p. 399-404.

I claim:

1. A human stem cell feeder layer cell line comprising an expression vector encoding senescence-triggering factors.

2. The cell line of claim 1, wherein the senescence triggering factors are Cip/Kip cyclin-dependent kinase inhibitor family members p21, p27, or p57

3. The cell line of claim 1, wherein the senescence triggering factors are INK4 cyclin-dependent kinase inhibitor family members p16, p18, or p19;

4. The cell line of claim 1, wherein the expression vector encodes one or a multiplicity of Cy motifs having an amino acid sequence (Lys/Arg)-Xaa-Leu, where Xaa is any amino acid.

5. The cell line of claim 1, wherein the expression vector encodes one or a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid.

6. The cell line of claim 1, capable of supporting growth of an embryonic stem cell.

7. The cell line of claim 1, capable of supporting growth of a fetal stem cell.

8. The cell line of claim 1, capable of supporting growth of an adult stem cell.

9. The cell line of claims 4 wherein the Cy motifs encode a peptide having an amino acid sequence identified by SEQ ID Nos. 3, 4, 5, 6, 7, 8, 9, 10, 11, 12 or 13.

10. The cell line of claims 4 or 9 wherein the expression vector encodes a multiplicity of Cy motifs having the same or different amino acid sequences and wherein the multiplicity of Cy motifs are expressed as a peptide multimer of said motifs.

11. The cell line of claim 1, wherein the expression vector encodes one or a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid.

12. The cell line of claims 11, wherein the expression vector encodes a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid, wherein the multiplicity of Ankyrin repeat motifs are expressed as a peptide multimer of said motifs.

13. A method of producing a human stem cell feeder cell layer for growing stem cells in culture by introducing into the cells of the human stem cell feeder cell layer expression vectors producing senescence-triggering factors, wherein premature senescence is induced in the human stem cell feeder layer by inducing expression of the senescence-triggering factors in the human stem cell feeder layer cells.

14. The method of claim 13, wherein the senescence triggering factors are Cip/Kip cyclin-dependent kinase inhibitor family members p21, p27, or p57

15. The method of claim 13, wherein the senescence triggering factors are INK4 cyclin-dependent kinase inhibitor family members p16, p18, or p19;

16. The method of claim 13, wherein the expression vector encodes one or a multiplicity of Cy motifs having an amino acid sequence (Lys/Arg)-Xaa-Leu, where Xaa is any amino acid, and wherein the multiplicity of Cy motifs are expressed as a peptide multimer of said motifs.

17. The method of claim 13, wherein the expression vector encodes one or a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid, wherein the multiplicity of Ankyrin repeat motifs are expressed as a peptide multimer of said motifs.

18. The method of claim 13, wherein the stem cell feeder layer cell is capable of supporting growth of an embryonic stem cell.

19. The method of claim 13, wherein the stem cell feeder layer cell is capable of supporting growth of a fetal stem cell.

20. The method of claim 13, wherein the stem cell feeder layer cell is capable of supporting growth of an adult stem cell.

21. The method of claims 16, wherein the Cy motifs encode a peptide having an amino acid sequence identified by SEQ ID Nos. 3, 4, 5, 6, 7, 8, 9, 10, 11, 12 or 13.

22. The method of claims 16 or 21, wherein the expression vector encodes a multiplicity of Cy motifs having the same or different amino acid sequences.

23. The cell line of claim 1, wherein the expression vector comprising: (a) a minimal promoter comprising a TATA sequence; (b) two phased operators downstream from the TATA sequence; and (c) two phased operators upstream of the TATA sequence.

24. The method of claim 13, wherein the expression vector comprising: (a) a minimal promoter comprising a TATA sequence; (b) two phased operators downstream from the TATA sequence; and (c) two phased operators upstream of the TATA sequence.

25. A method for growing stem cells in culture comprising the step of growing the cells on a human stem cell feeder layer according to claim 1.

26. The method of claim 25, wherein the stem cells are human stem cells.

27. The method of claim 26, wherein the human stem cells are embryonic stem cells, fetal stem cells or adult stem cells.

28. A method for growing stem cells in culture comprising the step of growing the stem cells in a media conditioned by growth of a human stem cell feeder cell line according to claim 1.

29. The method of claim 28, wherein the stem cells are human stem cells.

30. The method of claim 29, wherein the human stem cells are embryonic stem cells, fetal stem cells or adult stem cells.

31. The cell line of claim 1, that is a fibroblast cell line.

32. The method of claims 13, 25 or 28, wherein the human stem cell feeder cell line is a fibroblast cell line.

33. A cell line according to claim 1, wherein the expression vectors wherein the expression vector encodes one or a multiplicity of Cy motifs having an amino acid sequence (Lys/Arg)-Xaa-Leu, where Xaa is any amino acid, and wherein the multiplicity of Cy motifs are expressed as a peptide multimer of said motifs, and wherein the expression vector encodes one or a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid, wherein the multiplicity of Ankyrin repeat motifs are expressed as a peptide multimer of said motifs.

34. A cell line according to claim 33, wherein the one or multiplicity of Cy motifs and one or multiplicity of Ankyrin repeat motifs are encoded in the same expression vector.

35. A method according to claim 13, 25 or 28, wherein the expression vectors wherein the expression vector encodes one or a multiplicity of Cy motifs having an amino acid sequence (Lys/Arg)-Xaa-Leu, where Xaa is any amino acid, and wherein the multiplicity of Cy motifs are expressed as a peptide multimer of said motifs, and wherein the expression vector encodes one or a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid, wherein the multiplicity of Ankyrin repeat motifs are expressed as a peptide multimer of said motifs.

36. A method according to claim 35, wherein the one or multiplicity of Cy motifs and one or multiplicity of Ankyrin repeat motifs are encoded in the same expression vector.

37. A conditioned cell culture media for growing stem cells in the presence of absence of a feeder cell layer, comprising culture media obtained after growth in the media of a stem cell feeder cell line according to claim 1.


SEQUENCE LISTING (25 sequences)

SEQ ID NO: 1 - protein, 40 aa, Escherichia coli
MSITKDQIIEAVAAMSVMDVVELISAMEEKFEVSAAAAVA

SEQ ID NO: 2 - DNA, 23 bp, Homo sapiens
aggatgttataaagcatgagtca

SEQ ID NO: 3 - protein, 6 aa, Artificial
  Synthetic; Cy motif of E2F2
KRRLDL

SEQ ID NO: 4 - protein, 6 aa, Artificial
  Synthetic; Cy motif of E2F3
KRRLEL

SEQ ID NO: 5 - protein, 6 aa, Artificial
  Synthetic; Cy motif of p107
KRRLFG

SEQ ID NO: 6 - protein, 6 aa, Artificial
  Synthetic; Cy motif of p130
KRRLFV

SEQ ID NO: 7 - protein, 6 aa, Artificial
  Synthetic; Cy motif of Cdc6
GRRLVF

SEQ ID NO: 8 - protein, 6 aa, Artificial
  Synthetic; Cy motif of Myt1
PRNLLS

SEQ ID NO: 9 - protein, 6 aa, Artificial
  Synthetic; Cy motif of Cdc25a
RRRLLF

SEQ ID NO: 10 - protein, 6 aa, Artificial
  Synthetic; Cy motif of p57
CRSLFG

SEQ ID NO: 11 - protein, 6 aa, Artificial
  Synthetic; Cy motif of p27
CRNLFG

SEQ ID NO: 12 - protein, 7 aa, Artificial
  Synthetic; Cy motif of N terminus of p21
NCRRLFG

SEQ ID NO: 13 - protein, 6 aa, Artificial
  Synthetic; Cy motif of C terminus of p21
KRRLIF

SEQ ID NO: 14 - protein, 6 aa, Artificial
  Synthetic; Cy motif of HPV18E1
KRRLFT

SEQ ID NO: 15 - protein, 6 aa, Artificial
  Synthetic; Cy motif of SSeCKS(1)
LKKLFS

SEQ ID NO: 16 - protein, 6 aa, Artificial
  Synthetic; Cy motif of SSeCK(2)
LKKLSG

SEQ ID NO: 17 - protein, 6 aa, Artificial
  Synthetic; Cy motif of b3-endonexin
KRSLKL

SEQ ID NO: 18 - protein, 6 aa, Artificial
  Synthetic; Cy motif of E2F1
KRRLDL

SEQ ID NO: 19 - protein, 21 aa, Artificial
  Synthetic; Ankyrin repeat motif of p16
RPVHDAAREGFLDTLVVLHRA

SEQ ID NO: 20 - protein, 21 aa, Artificial
  Synthetic; Ankyrin repeat motif of p19
SPVHDAARTGFLDTLKVLVEH

SEQ ID NO: 21 - protein, 21 aa, Artificial
  Synthetic; Ankyrin repeat motif of p18
AVIHDAARAGFLDTLQTLLEF

SEQ ID NO: 22 - protein, 21 aa, Artificial
  Synthetic; Ankyrin repeat motif of p15
RPVHDAAREGFLDTLVVLHRA

SEQ ID NO: 23 - protein, 18 aa, Artificial
  Synthetic; amino acid sequence of Ankyrin repeat motif
XXXHDAARXGFLDTLXXL

SEQ ID NO: 24 - protein, 6 aa, Artificial
  Synthetic; Cy motif of Cdc25a
PRRLLF

SEQ ID NO: 25 - protein, 6 aa, Artificial
  Synthetic; Cy motif of N terminus of p21
CRRLFG